(19) United States
(12) Patent Application Publication
(10) Pub. No.: US 2024/0207288 A1
(43) Pub. Date: Jun. 27, 2024

(54) METHODS OF TREATING ESOPHAGEAL STRICTURES

(71) Applicant: Ellodi Pharmaceuticals, L.P., Blue Bell, PA (US)

(72) Inventor: Gina EAGLE, Blue Bell, PA (US)

(21) Appl. No.: 18/287,416

(22) PCT Filed: Apr. 19, 2022
(86) PCT No.: PCT/US22/25297

§ 371 Date: Oct. 18, 2023

Related U.S. Application Data
(60) Provisional application No. 63/177,064, filed on Apr. 20, 2021.

(51) Int. Cl. A61K 31/56; A61K 9/00; A61K 9/20; A61P 1/00

(52) U.S. Cl. CPC A61K 31/56; A61K 9/0056; A61K 9/2077; A61P 1/00

(57) ABSTRACT

Disclosed herein are methods for topically treating strictures by orally administering corticosteroids. Dosages, formulations, and methods for administration of corticosteroids are provided.

### CROSS-REFERENCE TO RELATED APPLICATIONS

This application claims priority to and benefit of U.S. Provisional Application 63/177,064, filed Apr. 20, 2021, the entire contents of which are incorporated by reference.

### BACKGROUND

An esophageal stricture is an abnormal tightening or narrowing of the esophagus. A stricture narrows the esophagus, making it more difficult for food to travel down the tube. A stricture results from an injury to the esophagus that causes scarring and symptomatic narrowing of the esophagus. The main symptom of an esophageal stricture is dysphagia, and dysphagia is already major symptom of inflammatory diseases of the esophagus. Consequently, strictures can exacerbate dysphagia in patients with inflammatory diseases of the esophagus.

Strictures are notoriously difficult to treat. Currently, there are no treatments for strictures. Strictures also do not occur in all patients with inflammatory diseases of the esophagus. As a result, patients with severe strictures are generally excluded from treatment.

There exists a need in the art for methods of effectively treating strictures, while also controlling side effects such as fungal infections, including oral, oropharyngeal, and esophageal candidiasis. The present disclosure addresses these needs.

### SUMMARY OF THE INVENTION

The present disclosure provides methods for reducing the severity of strictures.

In some embodiments, the disclosure provides a method of topically treating an esophageal stricture in a patient in need thereof with a corticosteroid, comprising: (a) detecting the esophageal stricture; and then (b) orally administering the corticosteroid. In embodiments, about 1.5-3 mg of fluticasone propionate is administered. In embodiments, a corticosteroid is administered in an equipotent dose to about 1.5-3 mg of fluticasone propionate. In embodiments, a therapeutically effective amount of the oral corticosteroid contacts the esophageal stricture, thereby reducing the severity of the esophageal stricture. In embodiments, the detecting in step (a) comprises inserting an endoscope into the patient's esophagus. In embodiments, the endoscope has a diameter of about 9 mm. In embodiments, the esophageal stricture is detected visually using a video display on the endoscope. In embodiments, stricture is detected because the endoscope cannot pass through the patient's esophagus. In embodiments, the patient also has a ring. In embodiments, the ring is a grade 2 or grade 3 ring. In embodiments, the administrating the corticosteroid reduces the grade of the ring by at least 1 grade. In embodiments, the corticosteroid is administered while the patient is lying down or immediately prior to the patient lying down. In embodiments, wherein the patient is in a supine position. In embodiments, the corticosteroid is administered within about 5, 4, 3, 2, or 1 minutes prior to the patient lying down. In embodiments, the corticosteroid is administered at bedtime. In embodiments, the corticosteroid is administered about 30 minutes or less before target sleep time. In embodiments, the corticosteroid is administered once daily.

In some embodiments, the disclosure provides a method of topically treating an esophageal stricture in a patient in need thereof with a corticosteroid, comprising; orally administering the corticosteroid, wherein the stricture was detected visually using a video display on the endoscope or stricture was detected because the endoscope cannot pass through the patient's esophagus. In embodiments, the stricture was detected because the endoscope could not pass through the patient's esophagus. In embodiments, the endoscope has a diameter of about 9 mm. In embodiments, about 1.5-3 mg of fluticasone propionate is administered. In embodiments, a corticosteroid is administered in an equipotent dose to about 1.5-3 mg of fluticasone propionate. In embodiment, therapeutically effective amount of the corticosteroid contacts the esophageal stricture, thereby reducing the severity of the esophageal stricture. In embodiments, the patient also has a ring. In embodiments, the ring is a grade 2 or grade 3 ring. In embodiments, the administrating the corticosteroid reduces the grade of the ring by at least 1 grade. In embodiments, the corticosteroid is administered while the patient is lying down or immediately prior to the patient lying down. In embodiments, the patient is in a supine position. In embodiments, the corticosteroid is administered within about 5, 4, 3, 2, or 1 minutes prior to the patient lying down. In embodiments, the corticosteroid is administered at bedtime. In embodiments, the corticosteroid is administered about 30 minutes or less before target sleep time. In embodiments, the corticosteroid is administered once daily.

In some embodiments, the disclosure provides a method of treating strictures in a patient in need thereof comprising orally administering to the patient about 0.5 mg to about 6 mg of fluticasone propionate, or an equipotent dose of a corticosteroid, once daily for at least 12 weeks. In embodiments, during said 12 weeks: the patient's risk of candidiasis is less than about 10%, and the patient shows an improvement in at least one of the following outcomes compared to a patient that is administered the corticosteroid twice daily: (i) at least one symptom score measured using a patient reported outcome symptom evaluation (PROSE) instrument after an episode of dysphagia; (ii) EoE Endoscopic Reference (EREF) score; (iii) EoE Activity Index (EEsAI) avoidance, modification, and slow swallowing (AMS) score: (vi) Global EoE score; (v) Patient global impression of severity (PGIS); and (vi) patient global impression of change (PGIC).

In some embodiments, the symptom score is one or more of: (i) number of dysphagia free days over a 14-day period; (ii) the average daily episode severity score over a 14-day period; or (iii) the symptom burden over a 14-day period. In some embodiments, the symptom score is the number of dysphagia episodes over 14 days.

In some embodiments, after treating according to the methods of the disclosure, the patient shows an improvement in at least one of the following outcomes compared to a patient that is administered the corticosteroid twice daily: (i) at least one symptom score measured using a patient reported outcome symptom evaluation (PROSE) instrument after an episode of dysphagia; (ii) EoE Endoscopic Reference (EREFS) score; or (iii) Global EoE score.

In some embodiments, the methods of the disclosure comprise administering 1.5 mg, 3.0 mg, or 6 mg of fluticasone propionate, or an equipotent dose of a corticosteroid. In some embodiments, 3.0 mg of fluticasone propionate, or an equipotent dose of a corticosteroid is administered.

In some embodiments, fluticasone propionate or the corticosteroid is administered at bedtime or at nighttime.

In some embodiments, fluticasone propionate or the equipotent dose of the corticosteroid is administered while the patient is lying down or immediately prior to the patient lying down.

In some embodiments, the method of the disclosure reduces the patient's risk of candidiasis to 5% or less. In some embodiments, the candidiasis is oral candidiasis or esophageal candidiasis.

In some embodiments, the patient's risk of oral candidiasis is less than about 10%. In some embodiments, the patient's risk of oral candidiasis is less than about 4%, less than about 3%, less than about 2%, or less than about 1%. In some embodiments, according to the methods of the disclosure, a patient's risk of oral candidiasis is about 4.8%.

In some embodiments, the patient's risk of esophageal candidiasis is less than about 10%. In some embodiments, the patient's risk of esophageal candidiasis is about 9% or less, about 8% or less, about 7% or less, about 6% or less, or about 5% or less.

In some embodiments, the methods of the disclosure comprise utilizing a symptom score, wherein the symptom score comprises: (i) on a scale ranging from 0 to 10, a difficulty getting food down; (ii) on a scale ranging from 0 to 10, a worst discomfort with food; (iii) on a scale ranging from 0 to 10, a worst pain with food; (iv) a mean score of any combination of (i), (ii), and (iii); (v) a number of dysphagia episodes; (vi) a daily rate of dysphagia episodes; or (vii) a number of dysphagia-free days.

In some embodiments, the symptom score is the mean of two or more symptom score measurements. In some embodiments, the mean score is the mean of (i) on a scale ranging from 0 to 10, a difficulty getting food down, (ii) on a scale ranging from 0 to 10, a worst discomfort with food, and (iii) on a scale ranging from 0 to 10, a worst pain with food. In some embodiments, the mean score is calculated for one or more episodes of dysphagia over a period of time. For example, the mean score may be calculated from the score for each episode of dysphagia over 1 day, 2 days, 3 days, 4 days, 5 days, 6 days, 7 days, 8 days, 9 days, 10 days, 11 days, 12 days, 13 days, 14 days, or 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34, 35, 36, 37, 38, 39, 40 weeks, 41 weeks, 42 weeks, 43 weeks, 44 weeks, 45 weeks, 46 weeks, 47 weeks, 48 weeks, 49 weeks, 50 weeks, 51 weeks, or 52 weeks. In some embodiments, the mean score may be calculated from the score for each episode of dysphagia over 1, 2, 3, 4, 5, 6, 7, 8, 9, 10, 11, 12, 13, 14, 15, 16, 17, 18, 19, 20, 21, 22, 23, or 24 months. The mean score of (i) difficulty, (ii) discomfort and (iii) pain may be referred to as the “episode severity score.” An episode severity score may be assigned to a single episode of dysphagia. Alternatively, or in addition, an episode severity score may be assigned each day as the “daily episode severity score”. The daily episode severity score is the average episode severity score of all episodes of dysphagia that occur on a single day. In some embodiments, the daily episode severity score is averaged over a fourteen-day period. In some embodiments, the methods of the disclosure utilize the mean score of the symptom score, wherein if the patient experiences more than one episode of dysphagia, the mean score is calculated for the worst episode or worst symptoms of dysphagia. In some embodiments, the methods of the disclosure utilize the mean score of the symptom score, wherein the mean score is: (a) a daily mean of (i), (ii), and (iii) over a 14 day period; (b) a mean score of (i), (ii), and (iii) for the worst episode of dysphagia over a 14 day period; (c) a score for the worst symptom of dysphagia over a 14 day period; (d) a number of dysphagia episodes; (e) a daily rate of dysphagia episodes; or (f) a number of dysphagia-free days.

In some embodiments, the methods of the disclosure lead to an improvement in the symptom score by 0.5 to 4 points.

In some embodiments, the symptom score, the mean score, the worst episode score, or the worst symptom score is determined using data from 2 weeks of entries immediately prior to Week 12 and Week 26 of treatment.

In some embodiments, the methods of the disclosure lead to an improvement in the EREFS score by about 0.3 to 1.5 points.

In some embodiments, the methods of the disclosure lead to an improvement in the Global EoE score by about 1 to 4 points.

In some embodiments, the methods of the disclosure cause the PGIS score to shift to improvement by about 1 to 5 severity categories.

In some embodiments, the methods of the disclosure cause the EEsAI score to improve by about 2 to 15 points.

In some embodiments, the methods of the disclosure cause the patient to show improvement in the Eosinophilic Esophagitis Quality of Life Questionnaire (EoO-QoL-A).

In some embodiments, the methods of the disclosure cause the patient to show improvement by about 1 to 3 points in the Eosinophilic Esophagitis Quality of Life Questionnaire (EoO-QoL-A).

In some embodiments, the methods of the disclosure lead to a reduction in a patient's eosinophil count in the patient's esophagus compared to the patient's baseline eosinophil levels.

In some embodiments, the methods of the disclosure lead to an eosinophil count that is reduced to no more than 6 eosinophils per high power field (hpf).

In some embodiments, the methods of the disclosure involve measurement of the eosinophil count in the distal portion of the esophagus, the proximal portion of the esophagus, or both.

In some embodiments, the methods of the disclosure lead to an eosinophil count in the distal portion of the esophagus of no more than 6 eosinophils per hpf.

In some embodiments, the methods of the disclosure lead to an eosinophil count in the proximal portion of the esophagus of no more than 6 eosinophils per hpf.

In some embodiments, the methods of the disclosure lead to a decreased number of dysphagia episodes compared to a patient that is administered the corticosteroid twice daily.

In some embodiments, the methods of the disclosure lead to an increased number of dysphagia-free days compared to a patient that is administered the corticosteroid twice daily.

In some embodiments, the methods of the disclosure provide measurement of the risk of candidiasis and an improvement in at least one of the outcomes at week 1, 2, 3, 4, 5, 6, 7, 8, 9, 10, 11, and/or 12.

In some embodiments, the methods of the disclosure measure the risk of candidiasis and an improvement in at least one of the outcomes again at week 26 and/or week 52.

In some embodiments, the methods of the disclosure provide administration of a corticosteroid in an amount ranging from about 1 mg to about 5 mg, including 1 mg, 1.5 mg, 2 mg, 2.5 mg, 3, mg, 3.5 mg 4, mg, 4.5 mg, and 5 mg.

In some embodiments, fluticasone propionate or the corticosteroid is administered for about 12 weeks to at least one year.

In some embodiments, 1.5 mg of fluticasone propionate is administered. In some embodiments, 3.0 mg of fluticasone propionate is administered.

In some embodiments, the corticosteroid is budesonide. In some embodiments, budesonide is administered for about 12 weeks to at least one year. In some embodiments, 0.5-2 mg of budesonide is administered.

In some embodiments, the corticosteroid that is formulated as a solid composition. In some embodiments, the solid composition is in the form of a gel, lozenge, lollipop, effervescent tablet, powder, granules, an orally disintegrating composition or an orally dispersing composition. In some embodiments, the orally disintegrating composition is a tablet, wafer, film, effervescent, or lyophilized matrix. In some embodiments, the orally dispersing composition is a tablet, wafer, film, effervescent, or lyophilized matrix.

In some embodiments, the methods of the disclosure provide an improvement in the visual dysphagia question (VDQ) composite score compared to a patient that is administered the corticosteroid twice daily.

In some embodiments, the methods of the disclosure provide an improvement in the EEsAI total score compared to a patient that is administered the corticosteroid twice daily.

### BRIEF DESCRIPTION OF THE DRAWINGS

FIG. 1 illustrates the study design comparing Fluticasone Propionate (FP) Orally Disintegrating Tablet versus placebo in adults with eosinophilic esophagitis (“EoE”).

FIG. 2 illustrates endoscopy scans showing varying degrees of esophageal strictures.

FIG. 3 illustrates a representative bar graph of histological, symptomatic, and endoscopic response rates in the doing groups.

### DETAILED DESCRIPTION

Disclosed herein are methods of topically treating esophageal strictures and rings. These fibrostenotic features are not present in all patients with inflammatory esophageal disease, and therefore prior treatments of inflammatory esophageal diseases with oral corticosteroid do not necessarily treat fibrostenotic features, such as strictures. Furthermore, fibrostenotic features are are notoriously difficult to treat. In fact, patients with these fibrostenotic features, particularly severe strictures, are generally excluded from clinical trials. Applicants surprisingly and unexpectedly discovered that orally administering a corticosteroid according to the methods disclosed herein not only reduces strictures and rings. In some embodiments, once daily administration (e.g., at night, e.g., while lying down or immediately before lying down) results in improved treatment of fibrostenotic features compared to a patient that was treated with the corticosteroid twice daily. Surprisingly, the once-daily administration also reduces the risk of candidiasis and improves symptom scores as measured herein. Thus, once-daily treatment results in a better clinical outcome compared to twice-daily treatment. In some embodiments, the total daily dose for the once-daily administration and for the twice-daily administration is the same. For example, a patient who received 3 mg fluticasone propionate once-daily according to the methods describes herein has improved symptom scores and reduced risk of candidiasis compared to a patient that received 1.5 mg twice daily (BID), for a total daily dose of 3 mg. In some embodiments, the total daily dose for twice daily administration is more than the total daily dose for the once daily administration. For example, a patient that is administered 3 mg of a corticosteroid once daily at bedtime according to the methods describes herein has improved symptom scores and reduced candidiasis compared a patient receiving 3 mg of corticosteroid twice-daily (i.e., a total daily dose of 6 mg).

### Definitions

As used herein, and in the appended claims, the singular forms “a”, “an”, and “the” include plural referents unless the context clearly dictates otherwise. Thus, for example, reference to “a corticosteroid” can refer to one corticosteroid or to mixtures of corticosteroid, and reference to “the method” includes reference to equivalent steps and/or methods known to those skilled in the art, and so forth.

As used herein, the term “about” or “approximately” when preceding a numerical value indicates the value plus or minus an acceptable degree of variation in the art. In some embodiments, “about” indicates the value plus or minus a range of 10%. For example, “about 100” encompasses 90 and 110.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of EoE; ameliorating or reducing the development of or symptoms of EoE; or a combination thereof.

The terms “diagnose”, “diagnosis”, and “diagnosing”, as used herein, refer to identifying, assessing, evaluating, or classifying the presence or characteristics of a pathological condition, for example, diagnosing whether or not a subject referred to according to the method of the present disclosure has esophageal strictures or rings.

### Fibrostenotic Features and Methods of Treatment

In some embodiments, the present disclosure provides methods of treating fibrostenotic features in the esophagus, such as strictures or rings. Esophageal strictures represent fixed scaring of the esophagus secondary to prolonged inflammation. Esophageal strictures are often caused by a build-up of scar tissues from a variety of conditions or events that cause esophageal inflammation, including eosinophilic esophagitis (EoE), gastroesophageal reflux disease (GERD), endoscopy injury, extensive use of a nasogastric (NG) tube, swallowing of toxic substances, esophageal scleroderma, and during or following the treatment for esophageal varices. Rings refer to an abnormal ring of tissue in the esophagus that causes narrowing of the esophagus. Rings may be caused by the same conditions and events that cause strictures.

Several diagnostics may be used for detecting the fibrostenotic features esophageal strictures and/or rings. In embodiments, the esophageal strictures and/or rings may be detected by running an endoscope down the esophagus to search for rings or narrowing in the esophagus. In embodiments, esophagoscopy, esophageal manometry, modified barium swallow study (MBS), and/or flexible endoscopic evaluation of swallowing (FEES) may be utilized for detection of strictures or rings.

In embodiments, the esophageal stricture is detected by using an endoscope. In embodiments, the esophageal stricture is detected when the endoscope is inserted into the patient's esophagus but cannot pass through the patient's esophagus. In embodiments, the endoscope has a diameter ranging from about 7 mm to about 11 mm (for example, 7.1 mm, 7.2 mm, 7.3 mm, 7.4 mm, 7.5 mm, 7.6 mm, 7.7 mm, 7.8 mm, 7.9 mm, 8.0 mm, 8.1 mm, 8.2 mm, 8.3 mm, 8.4 mm, 8.5 mm, 8.6 mm, 8.7 mm, 8.8 mm, 8.9 mm, 9.0 mm, 9.1 mm, 9.2 mm, 9.3 mm, 9.4 mm, 9.5 mm, 9.6 mm, 9.7 mm, 9.8 mm, 9.9 mm, 10.0 mm, 10.1 mm, 10.2 mm, 10.3 mm, 10.4 mm, 10.5 mm, 10.6 mm, 10.7 mm, 10.8 mm, 10.9 mm, 11.0 mm, including all values and ranges therein). In embodiments the inserted endoscope has a diameter of about 8-10 mm. In embodiments the inserted endoscope has a diameter of about 9 mm. In embodiments, the endoscope provides a visual image or video displaying a portion or the entire esophagus. The esophageal stricture may be detected visually using a video display or by radiography.

In some embodiments, the strictures and/or rings are diagnosed using EoE Endoscopic Reference Score (EREFS), which measures edema (e.g., decreased vascularity or pallot), rings present, exudates (e.g., white plaques), furrows, and/or stricture in the esophagus according to Table 1. In embodiments, the patient also has a grade 2 or grade 3 ring.

TABLE 1
EREFS Score Assessment

| Characteristic | Grade 0 | Grade 1 | Grade 2 | Grade 3 |
|---|---|---|---|---|
| Edema (loss | Distinct | Decreased | Absent | |
| vascular | vascularity | | | |
| markings) | | | | |
| Rings | None | Mild | Moderate | Severe |
| (trachealization) | | (ridges) | (distinct | (cannot |
| | | | rings) | pass |
| | | | | endoscope) |
| Exudate | None | Mild (≤10% | Severe (≥10% | |
| (white | | surface area) | surface area) | |
| plaques) | | | | |
| Furrows | None | Mild | Severe | |
| (vertical | | | (depth) | |
| lines) | | | | |
| Stricture | Absent | Present | | |

Fibrostenotic features represent fixed scaring of the esophagus secondary to prolonged inflammation, and the methods of the disclosure are able to resolve the scaring. The resolution of scaring is independent of the condition or disease, and therefore the methods of the disclosure are suitable for use with any disease or condition in which fibrostenotic features occur. In some embodiments, the patient with the fibrostenotic feature has an inflammatory condition of the upper gastrointestinal tract, such as the esophagus. In some embodiments, the inflammatory condition is eosinophilic esophagitis (EoE), gastroesophageal reflux disease (GERD), non-erosive reflux disease (NERD), erosive esophagitis, Barrett's esophagus, eosinophilic gastroenteritis, hypereosinophilic syndrome, corrosive (caustic) chemical esophagitis, radiation-induced esophagitis, chemotherapy-induced esophagitis, transient drug-induced esophagitis (also known as medication esophagitis), persistent drug-induced esophagitis, Crohn's disease of the esophagus, post-surgical resection of the esophagus, and pseudomembranous esophagitis. In some embodiments, the patient has eosinophilic esophagitis (EoE). In some embodiments, the patient has gastroesophageal reflux disease (GERD). In some embodiments, the patient has non-erosive reflux disease (NERD). In some embodiments, the patient has erosive esophagitis. In some embodiments, the patient has Barrett's esophagus. In some embodiments, the patient has eosinophilic gastroenteritis. In some embodiments, the patient has hypereosinophilic syndrome. In some embodiments, the patient has corrosive (caustic) chemical esophagitis. In some embodiments, the patient has radiation-induced esophagitis. In some embodiments, the patient has chemotherapy-induced esophagitis. In some embodiments, the patient has transient drug-induced esophagitis (also known as medication esophagitis). In some embodiments, the patient has persistent drug-induced esophagitis. In some embodiments, the patient has Crohn's disease of the esophagus. In some embodiments, the patient has post-surgical resection of the esophagus. In some embodiments, the patient has pseudomembranous esophagitis. In some embodiments, the present disclosure includes a method for treating a patient having one or more of the above gastrointestinal disorders, wherein the patient also has a lactose allergy and/or a starch allergy.

In some embodiments, the corticosteroid disclosed herein is administered until the strictures and/or rings are treated. In some embodiments, the corticosteroid disclosed herein continues to be administered after the strictures and/or rings are treated.

In some embodiments, after oral administration, the corticosteroid contacts and/or is deposited on or near the esophageal fibrostenotic feature. In some embodiments, the corticosteroid contacts and/or is deposited in the distal portion of the esophagus. In some embodiments, the pharmaceutical composition contacts and/or is deposited in the proximal portion of the esophagus. In some embodiments, therapeutically effective amounts of the corticosteroid contacts and/or is deposited the distal and proximal portion of the esophagus.

Treatment of strictures and rings may be measured by any means known in the art. For example, treatment can be assessed by visual inspection using an endoscope equipped with a video display. Tests used to evaluate patients with esophageal inflammation such as EoE include, but are not limited to, biopsies, evaluation of symptoms (e.g. through patient reported outcome (PRO) or physician questionnaire), quality of life measurements, determination of Dysphagia-Free-Days in a patient, endoscopy (e.g. EREFS), esophageal compliance and/or improvement in esophageal remodeling (e.g. using a suitable diagnostic test such as EndoFLIP, available from Crospon Inc.), evaluation of biomarkers, decrease in peak eosinophil count, decrease in food impaction, EEsAI, Strong Dysphagia Index (DSQ), MDQ-30, EoE-QOL-A, VDQ (Visual Dysphagia Questionnaire), Avoidance Modification and Slow Eating (AMS) scores, and/or histology.

### Eosinophilic Esophagitis (EoE)

In some embodiments, the methods of the present disclosure are utilized to treat fibrostenotic features (strictures and/or rings) in a patient with EoE. Not all patients with EoE have esophageal strictures. Generally, EoE is not diagnosed by imaging studies alone and some patients with EoE report normal esophagus during the barium swallow testing. Although featureless narrow-caliber esophagus, ringed esophagus, and isolated esophageal stricture are sometimes seen in EoE, however, none of them are categorized as pathognomonic of EoE (Ahmed, M. et al., Eosinophilic esophagitis in adults, World J Gastrointest Pharmacol Ther, 2016 7 (2): 207-231).

EoE has been described in children and adults with dysphagia and other oesophageal symptoms either alone (typical presentation) or as a manifestation of eosinophilic gastroenteritis (unusual presentation). In its isolated form, the disease exhibits symptoms and histologies similar to gastroesophageal reflux disease (GERD) (e.g., dysphagia, food impaction, nausea, vomiting, and weight loss) and due to this in its first appearance in the 1960's EoE was originally diagnosed as GERD (Furuta et al. 2007). Over time, the similarities were questioned as EoE patients did not experience reflux and did not typically respond to anti-reflux therapy, and it was thereafter considered as a separate clinical entity.

EoE affects all ages and ethnic backgrounds. EoE is predominant in non-Hispanic whites. The majority of affected patients with EoE are male, who usually present with EoE symptoms during childhood or in their 30's or 40's. EoE in children usually presents between the ages of 5 and 10 years old and 70% of childhood EoE persists into adulthood. Clinical manifestations of EoE may vary with age with a difference in symptoms between infants and young children compared to adolescents and adults. In contrast to younger children, older children typically present with either heartburn or symptoms of dysphagia. Adolescents present with an oesophageal food impaction. Patients with an atopic background or food-allergies have been shown to present with more severe oesophageal symptoms and food impaction. In some embodiments, common symptoms of EoE during adolescence are GERD-like symptoms. In some embodiments, children aged 11 years and older mostly report dysphagia and food impactions, which are also the most common indications for endoscopy in adult patients.

In some embodiments, EoE is defined as a primary clinicopathologic disorder of the oesophagus, which is characterised by oesophageal and/or upper gastrointestinal (GI) tract symptoms in association with oesophageal mucosal biopsy specimens containing >15 intraepithelial eosinophils (EOS)/high power field (HPF) in one or more biopsy specimens and absence of pathologic GERD as evidenced by a normal pH monitoring study of the distal oesophagus or lack of response to high-dose proton pump inhibitor (PPI) medication.

In some embodiments, EoE patients may be diagnosed using any appropriate measures in the art. In some embodiments, the patient is diagnosed with EoE based on symptoms, score in the assessment using a patient reported outcome (PRO) questionnaire, histology, and/or failed documentation on proton pump inhibitors. In some embodiments, the patient received proton-pump inhibitor (PPI) therapy prior to administration of a corticosteroid. In some embodiments, the patient failed to improve after 8 weeks of high-dose (e.g., 40 mg) PPI. A lack of response to PPI therapy may be defined as Peak eosinophil count >15/HPF in at least one biopsied location after 8 weeks of treatment with a high dose PPI. In some embodiments, the failure of PPI therapy is documented before administration of a pharmaceutical composition of the present disclosure. In some embodiments, the patient did not receive PPI therapy prior to administration of a corticosteroid.

The present inventors surprisingly and unexpectedly found that once daily administration of a corticosteroid is more effective to treat strictures and/or rings in EoE symptoms than twice daily administration, even when the total daily dose for the once daily administration is the same as the total daily dose for twice daily administration. Not only is once daily administration of a corticosteroid effective to treat EoE (e.g., by reducing eosinophils), but once daily administration significantly reduces the risk of candidiasis compared to twice daily administration. As such, patients administered the corticosteroid once daily have surprisingly improved symptom outcomes compared to patients receiving twice daily administration.

### Corticosteroids and Additional Therapeutic Agents

In some embodiments, methods of the present disclosure involve the administration of one or more corticosteroids to a patient with esophageal strictures or rings. Suitable corticosteroids include, but are not limited to hydrocortisone, prednisone, prednisolone, methylprednisolone, dexamethasone, betamethasone, etc. or mineralocorticoid potencies (e.g., alsosterone), budesonide, fluticasone, flunisolide, ciclesonide, mometasone, beclomethasone, tixocortol and salts, or esters and mixtures thereof. In some embodiments, the corticosteroid is budesonide. In some embodiments, the corticosteroid is fluticasone, or an ester thereof. In some embodiments, the corticosteroid is fluticasone propionate.

Fluticasone propionate (FP) is a medium-potency synthetic corticosteroid having the chemical name S-(fluoromethyl)-6α,9-difluoro-11β, 17-dihydroxy-16α-methyl-3-oxoandrosta-1, 4-diene-17β-carbothioate, 17-propanoate. The molecular formula of fluticasone propionate is C₂₅H₃₁F₃O₅S. The chemical structure of fluticasone propionate is:

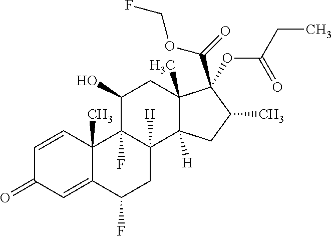

Fluticasone propionate (also referred to herein as “FP”) is a white to off-white powder. It is freely soluble in dimethyl sulfoxide and dimethylformamide, sparingly soluble in acetone, dichloromethane, ethyl acetate and chloroform, slightly soluble in methanol and 95% ethanol, and practically insoluble in water. FP decomposes without melting. The onset of decomposition occurs at about 225° C. Fluticasone propionate is a medium-potency glucocorticoid with anti-inflammatory including anti-eosinophilic activity in vitro and in vivo.

In some embodiments, the pharmaceutical compositions used in (or for use in) the methods described herein can be any dosage form which can topically administer a corticosteroid to the esophagus. Non-limiting examples of suitable dosage forms include liquid compositions (e.g., solutions, suspensions, and slurries), gels, and solid compositions which form a liquid or gel after oral administration. For example, orally disintegrating compositions (e.g., ODT, effervescent, film, lyophilize matrix, or wafer), lozenges, and lollipops can from a solution, suspension, or gel comprising the therapeutic agent in the oral cavity of the patient, and after the solution or suspension is swallowed, the corticosteroid dissolved or suspended therein contacts the esophagus as the liquid traverses the esophageal tract. In a preferred embodiment, the pharmaceutical composition is in the form of an ODT.

Wafers can include dried or lyophilized compositions such as orally disintegrating or dissolving dosage forms prepared using Zydis® lyophilization technology (e.g., as described in U.S. Pat. No. 6,316,027), containing a corticosteroid as the active pharmaceutical ingredient. Film dosage forms can include edible films such as those described in U.S. Pat. No. 6,596,298 or U.S. Pat. No. 6,740,332, containing a corticosteroid as the active pharmaceutical ingredient. In some embodiments, the solid composition comprises a lyophilized matrix, wherein the lyophilized matrix comprises a corticosteroid, the carrier and excipient. Suitable excipients include, but are not limited to, mannitol, xylitol, sorbitol, maltol, maltitol, lactose, sucrose, maltose, and combinations thereof.

Effervescent tablets and effervescent orally dispersing tablets can include those disclosed in U.S. Pat. Nos. 9,867,780, 8,580,300. Such formulations contain weak acids or salts of weak acids, such as tartaric acid, acetic acid, lactic acid, or citric acid, or pharmaceutically acceptable salts thereof, such as magnesium, calcium, or sodium salts. These formulations may also include pharmaceutically acceptable excipients that release CO₂ upon contact with water (e.g., saliva), such as carbonic acid, and salts of carbonates and bicarbonates, such as sodium and potassium salts. In some embodiments, such effervescent tablets are formulated to dissolve in a solution prior to oral administration. Such formulations may further comprise polyvinylpyrrolidone.

In some embodiments, fluticasone propionate is formulated as an orally disintegrating (also referred to as orally dispersing or orodispersible) tablets with an excipient mixture consisting of crospovidone, mannitol colloidal silicon dioxide, silicified microcrystalline cellulose, sucralose, and sodium stearyl fumarate. In some embodiments, the orally disintegrating table comprises about 1.5 or 3.0 mg of fluticasone propionate. In some embodiments, the ODT is described in U.S. Pat. No. 8,771,729 or U.S. Pat. No. 10,471,071, each of which are herein incorporated by reference.

In some embodiments, one or more additional therapeutic agents may be co-administered with the corticosteroid. Such therapeutic agents include proton pump inhibitors (PPI), including, but not limited to, omeprazole, lansoprazole, dexlansoprazole, rabeprazole, pantoprazole, and esomeprazole.

In some embodiments, the additional therapeutic agent comprises one or more immunosuppressant. Suitable immunosuppressants include, but are not limited to, cyclosporine, tacrolimus, prednisolone, hydrocortisone, sirolimus, everolimus, azathioprine, mycophenolic acid, methotrexate, basiliximab, daclizumab, rituximab, mepolizumab (anti-IL-5), reslizumab (anti-IL-5), QAX576 (anti-IL-13), omalizumab (anti-immunoglobulin-E), infliximab (anti-TNF-α), anti-thymocyte globulin, and anti-lymphocyte globulin.

In some embodiments, the pharmaceutical compositions disclosed herein are co-administered with one or more antibodies. Suitable anti-bodies include, include IL-4, IL-5, and IL-13 antibodies. Non-limiting examples include basiliximab, daclizumab, rituximab, mepolizumab (anti-IL-5), reslizumab (anti-IL-5), QAX576 (anti-IL-13), and omalizumab (anti-immunoglobulin-E).

In some embodiments, the one or more therapeutic agents may be “co-administered”, i.e., administered together in a coordinated fashion to a subject, either as separate pharmaceutical compositions or admixed in a single pharmaceutical composition. By “co-administered”, the one or more therapeutic agents may also be administered simultaneously with the present pharmaceutical compositions, or be administered separately, including at different times and with different frequencies. The one or more therapeutic agents may be administered by any known route, such as orally, intravenously, intramuscularly, nasally, subcutaneously, and the like; and the therapeutic agent may also be administered by any conventional route.

In some embodiments, the therapeutic agents in the above paragraphs can be combined. When two or more medicines are used in combination, dosage of each medicine is commonly identical to the dosage of the medicine when used independently. If a medicine interferes with metabolism of other medicines, the dosage of each medicine is properly adjusted. Each medicine may be administered simultaneously or separately in an appropriate time interval.

In some embodiments, the therapeutic agent for use in the present methods can be formulated into any appropriate dosage form, such as oral orally, parenterally, by inhalation spray, or topically, in formulations containing pharmaceutically acceptable carriers, adjuvants and vehicles. The term parenteral as used here includes subcutaneous, intravenous, intramuscular, and intraarterial injections with a variety of infusion techniques.

### Dosing

In some embodiments, the methods of the disclosure involve administration of a therapeutically effective dose of fluticasone propionate. In some embodiments, the therapeutically effective dose is from about 0.5 mg to about 5 mg of fluticasone propionate. In some embodiments, fluticasone propionate is administered in a dose of about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, or about 5.0 mg. In some embodiments, 1.5 mg of fluticasone propionate or an equipotent dose of a corticosteroid is administered. In some embodiments, 3.0 mg of fluticasone propionate or an equipotent dose of a corticosteroid is administered.

While the compositions and methods described herein use or refer to a dose of fluticasone propionate, the disclosure envisions using other corticosteroids and obtaining substantially similar efficacy in treating fibrostenotic features. The disclose envisions that such corticosteroid are used in an equipotent dose to fluticasone propionate to achieve the efficacy. In embodiments, the corticosteroid has an equipotent dose to 1.5 mg or 3.0 mg of fluticasone propionate. A person of skill in the art could determine the equipotent dose based on the corticosteroid's relative glucocorticoid receptor binding affinity or the relative potency of the corticosteroid's anti-inflammatory activity. In some embodiments, the equipotent dose is calculated based on the relative glucocorticoid activity corticosteroid's relative glucocorticoid receptor binding affinity. The glucocorticoid receptor binding affinity gives a measure of the dose necessary to occupy 50% of the glucocorticoid receptors. Table 2 gives the relative glucocorticoid receptor binding affinities for a number of corticosteroids. In some embodiments, pharmacokinetic/pharmacodynamic modelling is utilized to estimate the equipotent dose of corticosteroid. The following article which describes methods of calculating equipotent doses is incorporated by reference in its entirety herein: Daley-Yates, Br J Clin Pharmacol. 2015 September; 80(3): 372-380.

TABLE 2
Relative Glucocorticoid Receptor Binding Affinities

| | Relative |
|---|---|
| | Glucocorticoid |
| | Receptor Binding |
| Drug | Affinity |
| Fluticasone furoate DPI | 2989 |
| Mometasone furoate DPI | 2100 |
| Fluticasone propionate DPI | 1775 |
| Beclomethasone dipropionate | 53 (1345) |
| (BMP) MDI | |
| Ciclesonide (des-CIC) MDI | 12 (1200) |
| Budesonide DPI | 935 |
| Triamicinolone acetonide MDI | 233 |
| Flunisolide MDI | 190 |
| Prednisolone oral | 12 |

MDI: metered-dose inhaler;
DPI: dry-powder inhaler

In some embodiments, the methods of the disclosure provide administration of a corticosteroid in an amount ranging from about 1 mg to about 5 mg, including 1 mg, 1.5 mg, 2 mg, 2.5 mg, 3, mg, 3.5 mg 4, mg, 4.5 mg, and 5 mg. In some embodiments, the equipotent dose of a corticosteroid is from 0.05 mg to 20 mg. In some embodiments, the equipotent dose of a corticosteroid ranges from about 0.05 mg to about 20 mg, e.g., about 0.05 mg, or about 0.1 mg, or about 0.15 mg, or about 0.2 mg, or about 0.25 mg, or about 0.30 mg, or about 0.35 mg, or about 0.40 mg, or about 0.45 mg, or about 0.50 mg, or about 0.55 mg, or about 0.60 mg, or about 0.65 mg, or about 0.70 mg, or about 0.75 mg, or about 0.80 mg, or about 0.85 mg, or about 0.9 mg, or about 0.95 mg, or about 1.0 mg, or about 1.5 mg, or about 2.0 mg, or about 2.5 mg, or about 3.0 mg, or about 3.5 mg, or about 4.0 mg, or about 4.5 mg, or about 5.0 mg, or about 5.5 mg, or about 6.0 mg, or about 6.5 mg, or about 7.0 mg, or about 7.5 mg, or about 8.0 mg, or about 8.5 mg, or about 9.0 mg, or about 9.5 mg, or about 10.0 mg, or about 10.5 mg, or about 11.0 mg, or about 11.5 mg, or about 12.0 mg, or about 12.5 mg, or about 13.0 mg, or about 13.5 mg, or about 14.0 mg, or about 14.5 mg, or about 15.0 mg, or about 15.5 mg, or about 16.0 mg, or about 16.5 mg, or about 17.0 mg, or about 17.5 mg, or about 18.0 mg, or about 19.0 mg, or about 19.5 mg, or about 20.0 mg, including all ranges between these values. Non-limiting examples of corticosteroids include hydrocortisone, prednisone, prednisolone, methylprednisolone, dexamethasone, betamethasone, etc. or mineralocorticoid potencies (e.g., alsosterone), budesonide, fluticasone, flunisolide, ciclesonide, mometasone, beclomethasone, tixocortol and salts, or esters and mixtures thereof.

In some embodiments, the methods of the disclosure involve administration of a total daily dose. As defined herein, the “total daily dose” is the total amount of fluticasone propionate or an equipotent dose of a corticosteroid administered in one day. As discussed herein, once-daily administration of a corticosteroids, according to the methods disclosed herein, treats fibrostenotic features, improves a patient's symptom scores (as described herein) while also reducing the patient's risk of candidiasis. The total daily dose for once-daily administration may be the same or different as the total daily dose for the twice-daily administration. In some embodiments, the total daily dose for twice daily administration is the same as the total daily dose of once daily administration. For example, the total daily dose for once-daily and twice-daily administration may be 1.5 mg or 3.0 mg fluticasone propionate. In some embodiments, the total daily dose for twice daily administration is more than the total daily dose for the once daily administration. For example, a patient may be administered 1.5 mg fluticasone propionate once-daily, and the patient's symptoms scores and risk of candidiasis may be compared to a patient that receives a total daily dose of 3.0 mg, 4.5 mg, or 6 mg fluticasone propionate, twice-daily. As another example, the patient may be administered 3 mg of a corticosteroid once-daily, and the patient's symptoms scores and risk of candidiasis may be compared to a patient that receives a total daily dose of 4.5 mg or 6 mg fluticasone propionate, twice-daily.

### Dosing Regimens

In some embodiments, the corticosteroid (e.g., fluticasone propionate) is administered once daily. In some embodiments, the corticosteroid (e.g., fluticasone propionate) is administered once a day at bedtime or nighttime (HS). As defined herein, bedtime is the time at which a patient desires to go to sleep. In some embodiments, the corticosteroid (e.g., fluticasone propionate) is administered within 30 minutes, or 1 hour, or 1.5 hours, or 2 hours, or 2.5 hours, or 3.0 hours of a patient's bedtime. In some embodiments, the corticosteroid (e.g., fluticasone propionate) is administered within 30 minutes of a patient's bedtime. In some embodiments, the corticosteroid (e.g., fluticasone propionate) is administered while the patient is lying down or immediately prior to the patient lying down. As used herein, “immediately prior to the patient lying down” means within 30 minutes of the patient lying down, e.g., within 25, 20, 15, 10 or 5 minutes of the patient lying down. In embodiments, the oral corticosteroid is administered within about 5, 4, 3, 2, or 1 minute prior to the patient lying down. In embodiments, the fluticasone propionate is administered within about 5, 4, 3, 2, or 1 minute prior to the patient lying down. In some embodiments, fluticasone propionate is administered once daily at about 6 p.m., about 6:30 p.m., about 7:00 p.m., about 7:30 p.m., about 8:00 p.m., about 8:30 p.m., about 9:00 p.m., about 9:30 p.m., about 10:00 p.m., about 10:30 p.m., about 11:00 p.m., or about 12:00 a.m. In some embodiments, fluticasone propionate is administered to a patient on an empty stomach (e.g., at least two hours after eating or at least one hour before eating; or at least 30 minutes before or after eating).

In some embodiments, the corticosteroid (e.g., fluticasone propionate) is administered for a defined length of time. In some embodiments, the methods of the disclosure provide administration of the corticosteroid (e.g., fluticasone propionate) is administered for about 12 weeks to at least one year. In some embodiments, the length of time is at least 12 weeks, or at least 13 weeks, or at least 14 weeks, or at least 15 weeks, or at least 16 weeks, or at least 17 weeks, or at least 18 weeks, or at least 19 weeks, or at least 20 weeks, or at least 21 weeks, or at least 22 weeks, or at least 23 weeks, or at least 24 weeks, or at least 25 weeks, or at least 26 weeks, or at least 27 weeks, or at least 28 weeks, or at least 29 weeks, or at least 30 weeks, or at least 31 weeks, or at least 32 weeks, or at least 33 weeks, or at least 34 weeks, or at least 35 weeks, or at least 36 weeks, or at least 37 weeks, or at least 38 weeks, or at least 39 weeks, or at least 40 weeks, or at least 41 weeks, or at least 42 weeks, or at least 43 weeks, or at least 44 weeks, or at least 45 weeks, or at least 46 weeks, or at least 47 weeks, or at least 48 weeks, or at least 49 weeks, or at least 50 weeks, or at least 51 weeks, or at least 52 weeks, or more (e.g., 1 year, 1.5 years, 2, years, 2.5 years, 3 years, 3.5 years, 4 years, 4.5 years, 5 years, and so on).

In some embodiments, the treatment of esophageal strictures or rings with a corticosteroid is stopped for a defined length of time to allow the patient to recover from treatment. In some embodiments, the length of time is at least 1 week, or at least 2 weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 13 weeks, or at least 14 weeks, or at least 15 weeks, or at least 16 weeks, or at least 17 weeks, or at least 18 weeks, or at least 19 weeks, or at least 20 weeks, or at least 21 weeks, or at least 22 weeks, or at least 23 weeks, or at least 24 weeks, or at least 25 weeks, or at least 26 weeks, or at least 27 weeks, or at least 28 weeks, or at least 29 weeks, or at least 30 weeks, or at least 31 weeks, or at least 32 weeks, or at least 33 weeks, or at least 34 weeks, or at least 35 weeks, or at least 36 weeks, or at least 37 weeks, or at least 38 weeks, or at least 39 weeks, or at least 40 weeks, or at least 41 weeks, or at least 42 weeks, or at least 43 weeks, or at least 44 weeks, or at least 45 weeks, or at least 46 weeks, or at least 47 weeks, or at least 48 weeks, or at least 49 weeks, or at least 50 weeks, or at least 51 weeks, or at least 52 weeks, or more.

In some embodiments, treatment is stopped for a defined length of time and then restarted. In some embodiments, treatment is restarted after 1 week, or at least 2 weeks, or at least 3 weeks, or at least 4 weeks, or at least 5 weeks, or at least 6 weeks, or at least 7 weeks, or at least 8 weeks, or at least 9 weeks, or at least 10 weeks, or at least 11 weeks, or at least 12 weeks, or at least 13 weeks, or at least 14 weeks, or at least 15 weeks, or at least 16 weeks, or at least 17 weeks, or at least 18 weeks, or at least 19 weeks, or at least 20 weeks, or at least 21 weeks, or at least 22 weeks, or at least 23 weeks, or at least 24 weeks, or at least 25 weeks, or at least 26 weeks, or at least 27 weeks, or at least 28 weeks, or at least 29 weeks, or at least 30 weeks, or at least 31 weeks, or at least 32 weeks, or at least 33 weeks, or at least 34 weeks, or at least 35 weeks, or at least 36 weeks, or at least 37 weeks, or at least 38 weeks, or at least 39 weeks, or at least 40 weeks, or at least 41 weeks, or at least 42 weeks, or at least 43 weeks, or at least 44 weeks, or at least 45 weeks, or at least 46 weeks, or at least 47 weeks, or at least 48 weeks, or at least 49 weeks, or at least 50 weeks, or at least 51 weeks, or at least 52 weeks, or more. Temporarily stopping the corticosteroid is a known as a drug “holiday”, and, in some embodiments, it may help to reduce cortisol suppression and other side effects associated with long-term corticosteroid use.

### Outcomes

In some embodiments, after administration of fluticasone propionate or an equipotent dose of a corticosteroid, the strictures are resolved. In some embodiments, the number patients with strictures after 12 weeks of treatment is reduced by 10%, 15%, 20%, 25%, 30%, 35%, 40%, 45%, 50%, 55%, 60%, 65%, 70%, 75%, 80%, 85%, 90%, 95, or 100%. In some embodiments, administration of fluticasone propionate or an equipotent dose of a corticosteroid reduces a ring grade by 1 or 2. In some embodiments, the patient develops no new strictures or rings during treatment. In some embodiments, change in ring grades and/or stricture severity are measured after 12 weeks of treatment.

In some embodiments, administration of fluticasone propionate or an equipotent dose of a corticosteroid once daily results in an improvement in one or more outcomes when compared to a patient administered fluticasone propionate or an equipotent dose of a corticosteroid twice daily. Non-limiting examples of patient outcomes include: a reduced risk or incidence of candidiasis; at least one symptom score measured using a patient reported outcome symptom evaluation (PROSE) instrument after an each episode of dysphagia (see e.g., WO 2019/165138) or the 24-hour diary (see e.g., US Publication No. 2016/0078186); the EoE Endoscopic Reference (EREF) score; the EoE Activity Index (EEsAI) avoidance, modification, and slow swallowing (AMS) score; the global EoE score; the eosinophilic oesophagitis quality of life questionnaire; the patient global impression of severity (PGIS); and the patient global impression of change (PGIC).

A patient's risk of candidiasis is determined from the incidence rate in the clinical trial population. The incidence rate of candidiasis is the number of patients that reported a candidiasis infection divided by the total number of patients treated with the corticosteroid during treatment.

In some embodiments, outcomes are measured 1 week, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 24 weeks, or 25 weeks, or 26 weeks, or 27 weeks, or 28 weeks, or 29 weeks, or 30 weeks, or 31 weeks, or 32 weeks, or 33 weeks, or 34 weeks, or 35 weeks, or 36 weeks, or 37 weeks, or 38 weeks, or 39 weeks, or 40 weeks, or 41 weeks, or 42 weeks, or 43 weeks, or 44 weeks, or 45 weeks, or 46 weeks, or 47 weeks, or 48 weeks, or 49 weeks, or 50 weeks, or 51 weeks, or 52 weeks, or 53 weeks, or 54 weeks, or 55 weeks, or 56 weeks, or 57 weeks, or 58 weeks, or 59 weeks, or 60 weeks, or 61 weeks, or 62 weeks, or 63 weeks, or 64 weeks, or 65 weeks, or 66 weeks, or 67 weeks, or 68 weeks, or 69 weeks, or 70 weeks, or 71 weeks, or 72 weeks, or 73 weeks, or 74 weeks, or 75 weeks, or 76 weeks, or 77 weeks, or 78 weeks, or 79 weeks, or 80 weeks, or 81 weeks, or 82 weeks, or 83 weeks, or 84 weeks, or 85 weeks, or 86 weeks, or 87 weeks, or 88 weeks, or 89 weeks, or 90 weeks, or 91 weeks, or 92 weeks, or 93 weeks, or 94 weeks, or 95 weeks, or 96 weeks, or 97 weeks, or 98 weeks, or 99 weeks, or 100 weeks, or 101 weeks, or 102 weeks, or 103 weeks, or 104 weeks, or more weeks after initiation of treatment according to the methods of the disclosure. In some embodiments, the methods of the disclosure result in an improvement in outcomes at after initiation of treatment according to the methods of the disclosure at 1 week, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 24 weeks, or 25 weeks, or 26 weeks, or 27 weeks, or 28 weeks, or 29 weeks, or 30 weeks, or 31 weeks, or 32 weeks, or 33 weeks, or 34 weeks, or 35 weeks, or 36 weeks, or 37 weeks, or 38 weeks, or 39 weeks, or 40 weeks, or 41 weeks, or 42 weeks, or 43 weeks, or 44 weeks, or 45 weeks, or 46 weeks, or 47 weeks, or 48 weeks, or 49 weeks, or 50 weeks, or 51 weeks, or 52 weeks, or 53 weeks, or 54 weeks, or 55 weeks, or 56 weeks, or 57 weeks, or 58 weeks, or 59 weeks, or 60 weeks, or 61 weeks, or 62 weeks, or 63 weeks, or 64 weeks, or 65 weeks, or 66 weeks, or 67 weeks, or 68 weeks, or 69 weeks, or 70 weeks, or 71 weeks, or 72 weeks, or 73 weeks, or 74 weeks, or 75 weeks, or 76 weeks, or 77 weeks, or 78 weeks, or 79 weeks, or 80 weeks, or 81 weeks, or 82 weeks, or 83 weeks, or 84 weeks, or 85 weeks, or 86 weeks, or 87 weeks, or 88 weeks, or 89 weeks, or 90 weeks, or 91 weeks, or 92 weeks, or 93 weeks, or 94 weeks, or 95 weeks, or 96 weeks, or 97 weeks, or 98 weeks, or 99 weeks, or 100 weeks, or 101 weeks, or 102 weeks, or 103 weeks, or 104 weeks, or more weeks. In some embodiments, the methods of the disclosure result in an improvement in any of the outcomes described herein for at least 52 weeks.

In some embodiments, the risk of candidiasis and an improvement in at least one of the outcomes are measured at week 1, 2, 3, 4, 5, 6, 7, 8, 9, 10, 11, and/or 12.

In some embodiments, the risk of candidiasis and an improvement in at least one of the outcomes again at week 26 and/or week 52 (or any week therebetween).

In some embodiments, the methods of the disclosure lead to a reduction in a patient's risk of candidiasis, a potentially adverse effect of oral steroid usage. In some embodiments, the methods of the disclosure provide an improvement in oral candidiasis, esophageal candidiasis, and/or oropharyngeal candidiasis. Oral, oropharyngeal, and esophageal candidiasis infections are known side effects of swallowed corticosteroids, such as budesonide and fluticasone propionate, used for the treatment of EoE. Oral candidiasis is one of the most common fungal infections affecting the fungal mucosa. Oral candidiasis is described by Agrawal et al. in the following citation Agrawal, A., Singh, A., Verma, R., & Murari, A. (2014). Oral candidiasis: An overview. Journal of Oral and Maxillofacial Pathology, 18(4), 81. doi:10.4103/0973-029x.141325; this reference is incorporated herein in its entirety. Oral candidiasis is caused by Candida albicans, Candida glabrata, Gandida guillermondii, Candida krusei, Candida guillermondii, Candida krusei, Candida parapsilosis, Candida pseudotropicalis, Candida stellatoidea, and Candida tropicalis. Candida can also infect the esophagus. Esophageal candidiasis is most commonly caused by Candida albicans. Esophageal candidiasis is detailed by Nishimura et al. in the following citation Nishimura, S., Nagata, N., Shimbo, T., Asayama, N., Akiyama, J., Ohmagari, N., Uemura, N. (2013). Factors Associated with Esophageal Candidiasis and Its Endoscopic Severity in the Era of Antiretroviral Therapy. PLoS ONE, 8(3). doi:10.1371/journal.pone.0058217 this reference is incorporated herein in its entirety. Without being bound by theory, it is postulated that patients who receive steroids experience candidiasis by possibly suppressing local cellular immunity and phagocytosis. The methods described herein reduce immune suppression. In some embodiments, according to the methods of the disclosure, a patient's risk of oral candidiasis is less than about 10% (e.g., about 10%, about 9%, about 8%, about 7%, about 6%, about 5%, about 4%, about 3%, about 2%, about 1% or less).

Candidiasis is diagnosed according to methods known to persons skilled in the art. In some embodiments, oral specimens are grown on agar. Briefly, specimens are collected under aseptic conditions from active lesions. Specimens are kept moist and stored in a refrigerator at 4° C. Smears are taken from the infected oral mucosa, rhagades, and fitting side of denture preferably with wooden spatulas. Smears were fixed immediately in ether/alcohol 1:1 or with spray fix. Dry preparations may be examined by Gram stain method and periodic acid Schiff (PAS) method. Swabs are seeded on various agar substrates to grow the yeast species. Pagano-Levin agar or Littman's substrate are useful supplements, because they enable distinction of yeasts on the basis of difference in colony color.

In some embodiments, an imprint culture technique is utilized for quantitative assessment of yeast growth in different areas of the oral mucosa. Sterile, square plastic foam pads are dipped in peptone water and placed on the restricted area under study for 30-60 seconds and placed thereafter directly on Pagano-Levin or Sabouraud's agar. Subsequently, candidal density at each site is determined by a Gallenkamp colony counter and expressed as colony forming units per mm² (CFU mm²). This technique is useful for localizing the site of infection.

In some embodiments, impression culture technique is used to estimate the number of colonies forming units of yeast. Maxillary and mandibular alginate impressions are taken and cast in 6% fortified agar, incorporated into Sabouraud's dextrose broth, and grown for 48-72 hours at 37° C., and the CFUs of yeast are estimated.

In some embodiments, the number of Candida in a patient's saliva is estimated by counting the resultant growth on Sabouraud's agar using either the spiral plating or Miles and Misra surface viable counting technique. Patients who display clinical signs of oral candidiasis usually have more than 400 CFU/mL.

In some embodiments, commercial identification kits are utilized to identify candidiasis, including the Microstix-candida system, the O Yeast-I dent system, and the Ricult-N dip slide technique.

In some embodiments, fungi in biopsy specimens are identified histologically. Hematoxylin and eosin poorly stain Candida species. The specific fungal stains such as PAS stain, Grocott-Gomori's metheneamine silver (GMS) and Gridley stains are widely used for demonstrating fungi in the tissues, which are colored intensely with these stains.

In some embodiments, physiological tests are used for definitive identification of Candida species. These tests involve the ability of the Candida species to assimilate and ferment individual carbon and nitrogen sources (see Table 3 and Table 4).

TABLE 3
Assimilation Reactions of Candida Species

| Candida species | Glu | Mal | Suc | Lac | Cel | Gal | Tre | Raff | Mel | Xyl | Ino | Dul |
|---|---|---|---|---|---|---|---|---|---|---|---|---|
| C. albicans | + | + | + | + | + | + | + | − | − | + | − | − |
| C. tropicalis | + | + | + | − | + | + | + | − | − | + | − | − |
| C. keyfer | + | − | + | + | + | + | − | + | − | + | − | − |
| C. parapsilosis | + | + | + | − | − | + | + | − | − | + | − | − |
| C. guilliermondii | + | + | + | − | + | + | + | + | − | + | − | + |
| C. krusei | + | − | − | − | − | − | − | − | − | + | − | − |

glucose (Glu), maltose (Mal), sucrose (Suc), lactose (Lac), cellobiose (Cel), galactose (Gal), trehalose (Tre), raffinose (Raff), melibiose (Mel), xylose (Xyl), inositol (Ino), and dulcitol (Dul)
+: Positive reaction,
−: Negative reaction

TABLE 4
Fermentation Reactions of Candida Species

| Candida Species | Glucose | Maltose | Sucrose | Lactose |
|---|---|---|---|---|
| C. albicans | AG | AG | − | − |
| C. tropicalis | AG | AG | AG | − |
| C. keyfer | AG | AG | AG | − |
| C. parapsilosis | AG | − | AG | − |
| C. guilliermondii | AG | − | − | − |
| C. krusei | AG | − | − | − |
| C. glabrata | AG | − | − | − |

+: Positive reaction,
−: Negative reaction,
A: Acid Production,
G: Gas production

In some embodiments, serological tests are utilized to detect invasive candidiasis including the detection of antibodies, immunodiffusion, slide agglutination, phytohemagglutination, coelectosynersis, immunoprecipitation, A and B immunofluorescence, nonspecific Candida antigens, latex agglutination, immunoblotting, 0-(1,3)-D-glucan, cell wall mannoprotein, cell wall components, and candida enolase antigen testing.

In some embodiments, an upper endoscopy is necessary for diagnosis, particularly if the candidiasis is an esophageal candidiasis. White-yellow plaques can be seen on upper endoscopy. Plaques and exudates are adherent to the mucosa and do not wash off with water irrigation. There may also be mucosal breaks or ulcerations. Hematoxylin and eosin stain of biopsies or brushing of esophageal candidiasis show pseudohyphae which is diagnostic for esophageal candidiasis. Pathology may demonstrate acute inflammation and/or intraepithelial lymphocytosis.

In some embodiments, patients treated according to the methods of the disclosure exhibit a risk of candidiasis of less than about 10%. In some embodiments, patients treated according to the methods of the disclosure exhibit a risk of candidiasis of less than about 9%, or less than about 8%, or less than about 7%, or less than about 6%, or less than about 5%, or less than about 4%, or less than about 3%, or less than about 2%, or less than about 1%. In some embodiments, the methods of the present disclosure lead to a reduced incidence of candidiasis. In some embodiments, incidences of candidiasis are reduced by about 1%, about 5%, about 10%, about 15%, about 20%, about 25%, about 30%, about 35%, about 40%, about 45%, about 50%, about 55%, about 60%, about 65%, about 75%, about 80%, about 85%, about 90%, about 95%, or about 100%, inclusive of all values and ranges therein, compared to an otherwise identical patient that is treated with the corticosteroid twice daily.

In some embodiments, according to the methods of the disclosure, a patient's risk of oral candidiasis is less than about 4%, less than about 3%, less than about 2%, or less than about 1%. In some embodiments, according to the methods of the disclosure, a patient's risk of oral candidiasis is about 4.8%. In some embodiments, instances of oral candidiasis are reduced by about 1%, about 5%, about 10%, about 15%, about 20%, about 25%, about 30%, about 35%, about 40%, about 45%, about 50%, about 55%, about 60%, about 65%, about 75%, about 80%, about 85%, about 90%, about 95%, or about 100%, inclusive of all values and ranges therein, compared to an otherwise identical patient that is treated with the corticosteroid twice daily.

In some embodiments, according to the methods of the disclosure, a patient's risk of esophageal candidiasis is less than about 10%. In some embodiments, according to the methods of the disclosure, a patient's risk of esophageal candidiasis is less than about 10%, or 9%, or 8%, or 7%, or 6%, or 5%, or 4%, or 3%, or 2%, or 1%. In some embodiments, instances of esophageal candidiasis are reduced by about 1%, about 5%, about 10%, about 15%, about 20%, about 25%, about 30%, about 35%, about 40%, about 45%, about 50%, about 55%, about 60%, about 65%, about 75%, about 80%, about 85%, about 90%, about 95%, or about 100%, inclusive of all values and ranges therein, compared to an otherwise identical patient that is treated with the corticosteroid twice daily.

In some embodiments, the methods of the present disclosure cause patients to show an improvement in at least one symptom score measured using a patient reported outcome evaluation (PROSE) instrument after an episode of dysphagia. The PROSE instrument computes several items, including the number of real-time episode entry (RTE) dysphagia episodes, the number of end of day recorded dysphagia episodes, the total number of dysphagia episodes, the proportion of RTE dysphagia episodes, the total duration of dysphagia, the total imputed duration of dysphagia, the number of dysphagia free days, the worst difficulty recorded in an RT episode, the worst pain recorded in an RT episode, the worst discomfort recorded in an RT episode, the worst composite symptom summary score, the worst difficulty recorded in an EOD episode, the worst pain recorded in an EOD episode, the worst discomfort recorded in an EOD episode, the worst composite symptom summary score, maximum reported difficulty response, maximum reported pain response, maximum reported discomfort response, and the worst composite symptom summary score. In some embodiments, PROSE computes the average of all ratings over a 14-day period. In some embodiments, the symptom score is the total number of dysphagia episodes experienced over 14 days. This may also be referred to as the frequency of dysphagia.

In some embodiments, PROSE provides symptom summary ratings, based on the following questions: (i) how difficult, on a scale from 1-10, was it for you to get the food and/or pills down? (ii) what was the worst pain you felt, on a scale from 1-10, when trying to get the food and/or pills down? (iii) What was the worst discomfort you felt, on a scale from 1-10, when trying to get the food/pills down? In some embodiments, the PROSE symptom score is the mean score of any combination of (i), (ii), and (iii). In some embodiments, the mean score of (i), (ii), and (iii) is referred to as “episode severity.”

An episode severity score may be assigned to a single episode of dysphagia. Alternatively, or in addition, an episode severity score may be assigned each day as the “daily episode severity score”. The daily episode severity score is the average episode severity score of all episodes of dysphagia that occur on a single day. In some embodiments, the daily episode severity score over a fourteen-day period is averaged.

In some embodiments, the methods of the disclosure lead to an improvement in the average daily episode severity score over a specific time period. The average daily episode severity score over a specific time period is the sum of the daily episode severity score for each day in which an episode of dysphagia is reported divided by the number of days in the time period that the episodes of dysphagia are reported. The episode severity score is the mean of the PROSE symptom scores (i), (ii), and (iii). In some embodiments, the average daily episode severity score is calculated over a time period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, 12 months, 18 months, or 2 years. In some embodiments, the average daily episode severity score is calculated over 14 days. For example, if the average daily episode severity score is calculated over a time period of 14 days and the patient experiences episodes of dysphagia on 12 out of 14 days of the time period, the average daily episode score over the 14-day time period is the sum of daily episode severity score of the 12 days reported divided by 12.

In some embodiments, the methods of the disclosure lead to an improvement in symptom burden. The symptom burden is the average daily episode severity score over a specific time period, including days in which no episodes of dysphagia are reported. As discussed herein, the daily episode severity score is the episode severity score divided by the number of dysphagia episodes in one day. The episode severity score is the mean of the PROSE symptom scores (i), (ii), and (iii), wherein a day with no dysphagia episodes is assigned a daily episode severity score of zero. In some embodiments, symptom burden is calculated over a time period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 10 weeks, 11 weeks, 12 weeks, 1 month, 2 months, 3 months, 4 months, 5 months, 6 months, 7 months, 8 months, 9 months, 10 months, 11 months, 12 months, 18 months, or 2 years. In some embodiments, the symptom burden is calculated over 14 days. For example, the symptom burden calculated over a time period of 14 days is the sum of the daily episode score of each of the 14 days divided by 14, wherein a day in which no dysphagia episodes are reported is assigned a daily episode score of 0.

In some embodiments, a daily episode severity score is the episode severity score for the worst episode of dysphagia. The worst episode of dysphagia in a given day has the highest episode severity score. The method to calculate the severity score id described herein.

In some embodiments, the methods of the disclosure lead to an improvement in the score of the worst symptom of dysphagia reported over a particular time period. In some embodiments, the worst symptom of dysphagia has the highest PROSE symptom score. For example, if a patient assigns (i) a score of 9, (ii) a score of 5, and (iii) a score of 1, (i) is the worst symptom.

In some embodiments, the PROSE symptom score includes the number of dysphagia episodes. In some embodiments, the PROSE symptom score includes the number of dysphagia episodes daily rate of dysphagia episodes. In some embodiments, the PROSE symptom score includes the number of dysphagia episodes daily rate of dysphagia episodes, a number of dysphagia-free days. In some embodiments, PROSE provides a daily mean composite score (e.g., for all reported incidences of dysphagia) over 14 days, a daily worst composite (e.g., for the reported incidences of dysphagia each day) score over 14 days, a daily worst composite score over 14 days, the number of episodes over 14 days, the daily rate of episodes over 14 days, or the number of dysphagia-free days over 14 days.

In some embodiments, the methods of the disclosure cause the PROSE score (e.g., the episode severity score described above) to improve by about 5%, or about 10%, or about 15%, or about 20%, or about 25%, or about 30%, or about 35%, or about 40%, or about 45%, or about 50%, or about 55%, or about 60%, or about 65%, or about 70%, or about 80%, or about 85%, or about 90%, or about 95%, or about 100%, or more. In some embodiments, the methods of the disclosure cause the number of episodes of dysphagia to decrease as determined by the PROSE instrument. In some embodiments, the methods of the disclosure cause the PROSE score to improve by about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, or more, inclusive of all ranges between these values.

In some embodiments, the PROSE instrument is described in International Publication Number WO/2019/165138, the contents of which are incorporated by reference herein in its entirety. The 24-hour diary refers to a device used for recording various events associated with dysphagia (e.g., associated with EoE) at the end of a 24-hour period, i.e., once a day. At the end of a 24-hour period, the patient recalls all the events associated with dysphagia that occurred over the previous 24-hour period, including inter alia, (i) the severity, intensity, duration, pain, discomfort, difficulty, and/or frequency of dysphagia, (ii) type (including dosage form and active agent) and timing of treatment, and (iii) avoidance measures. In some embodiments, the patient records entries in the 24-hour diary after the last meal. In some embodiments, the patient records entries in the 24-hour diary about 6 p.m., about 6:30 p.m., about 7:00 p.m., about 7:30 p.m., about 8:00 p.m., about 8:30 p.m., about 9:00 p.m., about 9:30 p.m., about 10:00 p.m., about 10:30 p.m., about 11:00 p.m., or about 12:00 a.m.

In some embodiments, methods of the present disclosure cause an improvement as suggested by the 24-hour diary outcome. U.S. Publication No. 2016/0078186 details the 24-hour diary outcome and is incorporated by reference in its entirety for all purposes.

In some embodiments, the methods of the present disclosure cause an improvement in a patient's EoE Endoscopic Reference score (EREFS). The EREFS identifies the severity of five endoscopic findings: edema, rings, exudates, furrows, and strictures. The EREFS classification system rates the severity of each of the endoscopic findings. The severity of edema is rated on a scale from 0 to 2. The severity of rings is rated from 0 to 3. The severity of exudates is rated from 0 to 2. The severity of furrows is rated from 0 to 2. The severity of strictures is rated from 0 to 1. The absence of a finding corresponds to a score of 0. The presence of a finding corresponds to a score of 1, 2, or 3. A higher score is correlated with higher severity. In some embodiments, the composite EREFS score, or the sum of the individual scores, is utilized to indicate the severity of EoE. In some embodiments, the inflammatory EREFS score, or the sum of the individual edeme, exudate, and furrows score, is utilized to indicate the severity of EoE. In some embodiments, a higher inflammatory or composite EREFS score corresponds to the severity of EoE. In some embodiments, the inflammatory or composite EREFS score decreases after treatment with a corticosteroid according to the methods of the disclosure. In some embodiments the inflammatory or composite EREFS score decreases by 0.1, or about 0.2, or about 0.3, or about 0.4, or about 0.5, or about 0.6, or about 0.7, or about 0.8, or about 0.9, or about 1.0, or about 1.1, or about 1.2, or about 1.3, or about 1.4, or about 1.5, or about 1.6, or about 1.7, or about 1.8, or about 1.9, or about 2.0, or about 2.1, or about 2.2, or about 2.3, or about 2.4, or about 2.5, or about 2.6, or about 2.7, or about 2.8, or about 2.9, or about 3.0, or about 3.1, or about 3.2, or about 3.3, or about 3.4, or about 3.5, or about 3.6, or about 3.7, or about 3.8, or about 3.9, or about 4.0, or about 4.1, or about 4.2, or about 4.3, or about 4.4, or about 4.5, or about 4.6, or about 4.7, or about 4.8, or about 4.9, or about 5.0, or about 5.1, or about 5.2, or about 5.3, or about 5.4, or about 5.5, or about 5.6, or about 5.7, or about 5.8, or about 5.9, or about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0, or about 8.1, or about 8.2, or about 8.3, or about 8.4, or about 8.5, or about 8.6, or about 8.7, or about 8.8, or about 8.9, or about 9.0, or about 9.1, or about 9.2, or about 9.3, or about 9.4, or about 9.5, or about 9.6, or about 9.7, or about 9.8, or about 9.9, or about 10.0 points, or more, inclusive of all ranges between these values. In some embodiments, the EREFS score decreases by 1%, or 5%, or 10%, or 15%, or 20%, or 25%, or 30%, or 35%, or 40%, or 45%, or 50%, or 55%, or 60%, or 65%, or 70%, or 75%, or 80%, or 85%, or 90%, or 95% or more, inclusive of all ranges between these values. The following article, incorporated by reference in its entirety herein, describes the EREFS score: Wechsler, Clin Hepatol. 2018 July; 16(7): 1056-1063.

In some embodiments, the patient's symptom score is evaluated using the Visual Dysphagia Question (VDQ). The VDQ addresses the severity of dysphagia when consuming food of 8 distinct consistencies. The 8 food consistencies and examples of foods to illustrate those consistencies are: 1) solid meat (such as steak, chicken, turkey, lamb), 2) soft foods (such as pudding, jelly, apple sauce), 3) dry rice or sticky Asian rice, 4) ground meat (hamburger, meatloaf), 5) fresh white untoasted bread or similar foods (such as doughnut, muffin, cake), 6) grits, porridge (oatmeal), or rice pudding, 7) raw fibrous foods (such as apple, carrot, celery), and 8) French fries. The degree of perceived difficulties when eating a given food consistency is graded between 0 for ‘No difficulties’ and 3 for ‘Severe difficulties’. A VDQ composite score is calculated using the individual grades for a given food consistency. The VDQ composite score is the sum of the grades for each food consistency divided by the maximum sum of individual grades for each food consistency that could be attained. The maximum sum of grades depends on the number of food consistencies consumed by a subject in a given recall period. In some embodiments, the VDQ composite score is improved after treating according to the methods of the disclosure. An improvement in the VDQ composite score is a decrease in VDQ composite score. In some embodiments, the VDQ composite score is improved by between about 1 and about 24 points, for example, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 points.

In some embodiments, outcomes of the methods of the disclosure are evaluated using the EoE Activity Index (EEsAI) avoidance modification, and slow swallowing (AMS) score. In some embodiments the EEsAI is improved by about 2 to 15 points. In some embodiments, the EEsAI score is improved by about 2.0, or about 2.1, or about 2.2, or about 2.3, or about 2.4, or about 2.5, or about 2.6, or about 2.7, or about 2.8, or about 2.9, or about 3.0, or about 3.1, or about 3.2, or about 3.3, or about 3.4, or about 3.5, or about 3.6, or about 3.7, or about 3.8, or about 3.9, or about 4.0, or about 4.1, or about 4.2, or about 4.3, or about 4.4, or about 4.5, or about 4.6, or about 4.7, or about 4.8, or about 4.9, or about 5.0, or about 5.1, or about 5.2, or about 5.3, or about 5.4, or about 5.5, or about 5.6, or about 5.7, or about 5.8, or about 5.9, or about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0, or about 8.1, or about 8.2, or about 8.3, or about 8.4, or about 8.5, or about 8.6, or about 8.7, or about 8.8, or about 8.9, or about 9.0, or about 9.1, or about 9.2, or about 9.3, or about 9.4, or about 9.5, or about 9.6, or about 9.7, or about 9.8, or about 9.9, or about 10.0, or about 10.1, or about 10.2, or about 10.3, or about 10.4, or about 10.5, or about 10.6, or about 10.7, or about 10.8, or about 10.9, or about 11.0, or about 11.1, or about 11.2, or about 11.3, or about 11.4, or about 11.5, or about 11.6, or about 11.7, or about 11.8, or about 11.9, or about 12.0, or about 12.1, or about 12.2, or about 12.3, or about 12.4, or about 12.5, or about 12.6, or about 12.7, or about 12.8, or about 12.9, or about 13.0, or about 13.1, or about 13.2, or about 13.3, or about 13.4, or about 13.5, or about 13.6, or about 13.7, or about 13.8, or about 13.9, or about 14.0, or about 14.1, or about 14.2, or about 14.3, or about 14.4, or about 14.5, or about 14.6, or about 14.7, or about 14.8, or about 14.9, or about 15 points, inclusive of all ranges between these values.

In some embodiments of the disclosure, the global EoE score is utilized to evaluate the outcomes of the methods of the disclosure. In some embodiments, the EoE score is improved by 1 point to 4 points. In some embodiments, the EoE score is improved by about 1 point, or about 2 points, or about 3 points, or about 4 points. In some embodiments, the EoE score is improved by 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%, or about 125%, or about 150%, or about 175%, or about 200%, or about 225%, or about 250%, or about 275%, or about 300% or more, inclusive of all ranges between these values.

In some embodiments, the adult EoE quality of life questionnaire (EoE-QoL-A) is utilized to evaluate the outcomes of the methods of the disclosure. The EoE-QoL-A) provides a measure of health-related quality of life. The EoE-QoL-A is a self-reported questionnaire designed to assess disease-specific health-related quality of life in adults with EoE. Questions are designed to evaluate established domains of health-related quality of life, including social functioning, emotional functioning, and disease impact on daily life experiences. The EoE-QoL-A includes 47 questions on a five-point scale. Higher scores indicate a better quality of life. In some embodiments, the methods of the present disclosure result in an improvement in the EoE-QoL-A. In some embodiments, the EoE-QoL-A score is improved by about 1 to about 3 points.

In some embodiments, the patient global impression of severity (PGIS) is utilized to evaluate the outcomes of the methods of the disclosure. The PGIS is a global index that may be used to rate the severity of EoE. The PGIS is measured on a scale of 1 to 7. A score of 1 corresponds to normal, and a score of 7 corresponds to extremely ill. A score of 4 corresponds to moderately ill. In some embodiments, the methods of the disclosure result in a reduction in PGIS score. In some embodiments, the PGIS score shifts to improvement by about 1 to 5 severity categories (e.g., about 1, 2, 3, 4 or 5 categories). In some embodiments, the PGIS is reduced by 1 point, or 2 points, or 3 points, or 4 points, or 5 points. In some embodiments, the PGIC is reduced by about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%, inclusive of all ranges between these values.

In some embodiments, the patient global impression of change (PGIC) is utilized to evaluate the outcomes of the methods of the disclosure. The PGIC is a global index that may be utilized to assess an improvement or a decline in clinical status. The PGIC is measured on a scale of 1 to 7. A score of 1 corresponds to very much improved, and a score of 7 corresponds to very much worse. A score of 4 corresponds to no change in a patient's symptoms. In some embodiments, the methods of the disclosure result in a reduction in PGIC score. In some embodiments, the PGIC is reduced by 1 point, or 2 points, or 3 points, or 4 points, or 5 points, or 6 points, inclusive of all ranges between these values. In some embodiments, the PGIC is reduced by about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%, inclusive of all ranges between these values.

In embodiments, a reduction in eosinophil count is associated with an improvement in EoE associated stricture. In some embodiments, the methods of the disclosure lead to a reduction in a patient's eosinophil count compared to the patient's baseline eosinophil levels. In some embodiments, the methods of the disclosure lead to an eosinophil count that is reduced to no more than 6 eosinophils per high power field (hpf). In some embodiments, the methods of the disclosure lead to an eosinophil count that is reduced to no more than 5 eosinophils per high power field (hpf), or 4 eosinophils per high power field (hpf), or 3 eosinophils per high power field (hpf), or 2 eosinophils per high power field (hpf), or 1 eosinophil per high power field (hpf). In some embodiments, the methods of the disclosure involve measurement of the eosinophil count in the distal portion of the esophagus, the proximal portion of the esophagus, or both. In some embodiments, the methods of the disclosure lead to an eosinophil count in the distal portion of the esophagus of no more than 6 eosinophils per hpf. In some embodiments, the methods of the disclosure lead to an eosinophil count in the distal portion of esophagus that is reduced to no more than 5 eosinophils per high power field (hpf), or 4 eosinophils per high power field (hpf), or 3 eosinophils per high power field (hpf), or 2 eosinophils per high power field (hpf), or 1 eosinophil per high power field (hpf). In some embodiments, the methods of the disclosure lead to an eosinophil count in the proximal portion of the esophagus of no more than 6 eosinophils per hpf. In some embodiments, the methods of the disclosure lead to an eosinophil count in the proximal portion of esophagus that is reduced to no more than 5 eosinophils per high power field (hpf), or 4 eosinophils per high power field (hpf), or 3 eosinophils per high power field (hpf), or 2 eosinophils per high power field (hpf), or 1 eosinophil per high power field (hpf).

In some embodiments, a symptom of EoE is dysphagia. In some embodiments, the methods of the disclosure lead to a decreased number of dysphagia episodes compared to a patient that is administered the corticosteroid twice daily. In some embodiments, the methods of the disclosure lead to an increased number of dysphagia-free days compared to a patient that is administered the corticosteroid twice daily

### EXAMPLES

### Example 1

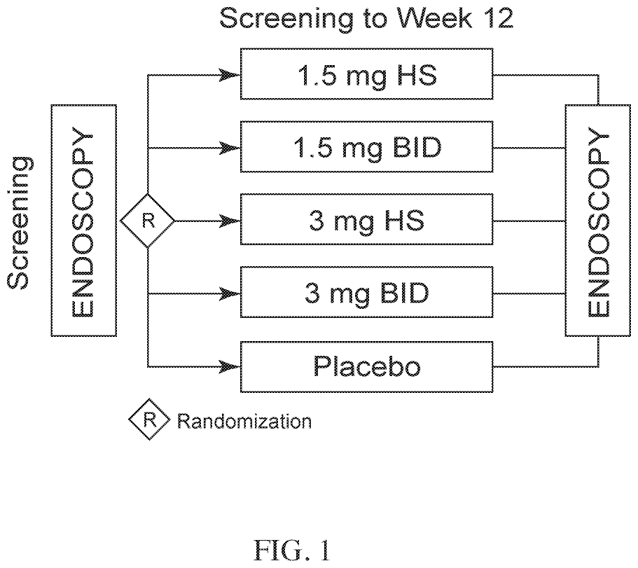

A clinical trial was performed to evaluate the effect of an orally disintegrating tablet (“ODT”) comprising fluticasone propionate (FP). A randomised, double-blind, placebo-controlled, multicentre, dose-ranging, and maintenance study of FP in subjects (>18 and <75 years of age) with strictures and/or rings and EoE. The trial examined four doses of FP to define the exposure-response of FP and the minimum effective dose to minimize significant hypothalamic-pituitary-adrenal (HPA) axis effects. The study (as shown in FIG. 1) was designed as follows.
(a) Screening: Patients with a confirmed diagnosis or presumptive diagnosis of EoE (including identifying strictures and/or rings) were selected for the study. An EoE diagnosis was confirmed by symptoms, histology, and historical documentation of failed treatment on >8 weeks of high-dose proton pump inhibitor (PPI). A high-dose PPI was defined as administration of a total dose of 20 to 40 mg of a marketed PPI once or twice daily. Summaries of the dosing group demographics are shown in Tables A. and B.

TABLE A
Dosing group demographics in the study

| | Randomized Dosing Group | | | | | | | | | | |
|---|---|---|---|---|---|---|---|---|---|---|---|
| | FP 3 | | FP 3 | | FP 1.5 | | FP 1.5 | | | | |
| | mg BID | | mg HS | | mg BID | | mg HS | | Placebo | | Total |
| Subject | (n = 20) | | (n = 21) | | (n = 22) | | (n = 21) | | (n = 19) | | (n = 103) |
| Completers (n, %) | 19 | (95.0%) | 20 | (95.2%) | 19 | 86.4%) | 18 | (85.7%) | 16 | (84.2%) | 92 (89.3%) |
| Male (n, %) | 16 | (80.0%) | 11 | (52.4%) | 15 | (68.2%) | 14 | (66.7%) | 16 | (84.2%) | 92 (89.3%) |
| Age (mean, years) | 36.8 | | 42.9 | | 41.3 | | 36.8 | | 38.6 | | 39.3 |
| BMI (kg/m²) | 25.54 | | 28.77 | | 27.39 | | 28.27 | | 28.36 | | 27.67 |
| History of esophageal | 8 | (40.0%) | 8 | (38.1%) | 10 | (45.5%) | 11 | (52.4%) | 9 | (47.4%) | 46 (44.7%) |
| stricture (n, %) | | | | | | | | | | | |
| Current esophageal | 4 | (20.0%) | 5 | (23.8%) | 5 | (22.7%) | 4 | (19.0%) | 5 | (26.3%) | 23 (22.3%) |

| | | | | | | |
|---|---|---|---|---|---|---|
| stricture | | | | | | |
| (mild-moderate) (n, %) | | | | | | |
| Baseline peak eosinophil | 55.1 ± 21.3 | 65.3 ± 28.9 | 69.2 ± 33.3 | 56.2 ± 25.9 | 64.3 ± 33.4 | 62.1 ± 29.0 |
| count | | | | | | |
| (mean eos/hpf ± SD) | | | | | | |
| Baseline endoscopic | 4.3 ± 2.9 | 5.1 ± 2.0 | 4.5 ± 2.1 | 5.1 ± 1.6 | 5.1 ± 1.7 | 4.8 ± 1.9 |
| severity | | | | | | |
| (mean EREFS ± SD) | | | | | | |
| Mean dysphagia | 14.3 ± 10.2 | 12.7 ± 5.9 | 16 ± 11.4 | 11.5 ± 5.7 | 13.9 ± 8.4 | 13.7 ± 8.6 |
| frequency | | | | | | |
| (over 14 days ± SD) | | | | | | |

TABLE B
Group demographics in the study including prior PPI treatment for EoE information

| | Randomized Dosing Group | | | | | | | | | |
|---|---|---|---|---|---|---|---|---|---|---|
| | FP 3 mg | | FP 3 mg | | FP 1.5 mg | | FP 1.5 mg | | | |
| | BID | | HS | | BID | | HS | | Placebo | |
| Subject | (n = 20) | | (n = 21) | | (n = 22) | | (n = 21) | | (n = 19) | |
| Age (mean, years) | 36.8 | | 42.9 | | 41.3 | | 36.8 | | 38.6 | |
| Male (n, %) | 16 | (80.0%) | 11 | (52.4%) | 15 | (68.2%) | 14 | (66.7%) | 14 | (73.7%) |
| BMI (kg/m²) | 25.54 | | 28.77 | | 27.39 | | 28.27 | | 28.36 | |
| Prior PPI treatment | 10 | (50%) | 12 | (57%) | 16 | (73%) | 9 | (43%) | 9 | (47%) |
| for EoE (n, %) | | | | | | | | | | |

(b) 4 week single-blind placebo run in/baseline symptom assessment: To assess baseline symptoms, all subjects that passed the screening part of the study received a placebo 30 minutes after breakfast and hora somni (before sleep; HS) (at bedtime).
(c) Part 1 (induction) (studies the effect of FP over 14 weeks): Patients that entered Part 1 of the study had evidence of EoE as defined by >15 peak eosinophils/TIPF. At least five to six biopsies should have been taken including both proximal and distal specimens.

Subjects that participated in Part 1 studies were randomized and administered a treatment selected from:
(a) 1.5 mg HS: Placebo 30 minutes after breakfast and 1.5 mg HS (at bedtime);
(b) 1.5 mg BID: 1.5 mg 30 minutes after breakfast and at bedtime; total daily dose of 3 mg;
(c) 3 mg HS: Placebo 30 minutes after breakfast and 3 mg at bedtime; and
(d) 3 mg BID: 3.0 mg 30 minutes after breakfast and at bedtime; total daily dose of 6 mg.

During Part 1 (Induction, Day 1 to Week 14), subjects received their randomized treatment for 14 weeks. At Week 12, the subjects underwent a response assessment, including an oesophagogastroduodenoscopy (EGD) to assess endoscopic and histologic status. Histologic responders and non-responders (at Week 12) entered Part 2.

Patient Population: 105 subjects received at least 1 dose of study drug in Part 1 of the study safety analysis set (SAF population), and 92 subjects (86.8%) of those subjects completed Week 14. The SAF population contained all subjects who were randomised and who did not meet any of the following criteria: subjects who did not receive any study drug, subjects given a wrong dose, or subjects mis-randomized. Subjects were classified according to their randomized treatment.

Primary Objective: The primary objective of the study was to evaluate the efficacy (histological response) of FP in adults with EoE.

Secondary Objectives: The secondary objective was to evaluate the effect of the ODT comprising FP on dysphagia episodes; to evaluate the change in EREFS, change in Global EoE Symptom Score and change in Dysphagia frequency.

### Endpoints:

The primary endpoint was the histologic responder rate at Week 12 of Part 1, defined as the percentage of subjects with <6 peak eosinophils/HPF after assessing at least 5 to 6 biopsies from the proximal and distal oesophagus (approximately 3 each) where the HPF area was 235 square microns (40 magnification lens with a 22 mm ocular).

The following secondary endpoints were evaluated through Week 12:
(a) EoE sustained response: Percentage of subjects who met the primary endpoint (histology) at Week 12 and maintained the primary endpoint at Weeks 26 and 52;
(b) Change from Baseline Eosinophilic Oesophagitis Endoscopic Reference Score (EREFS) at Weeks 12, 26, and 52: Endoscopic changes were as per the EREFS evaluation based on the following endoscopic features: Oedema, rings, exudates, furrows, stricture, and several miscellaneous features (crepe paper oesophagus, narrow calibre oesophagus, and oesophageal erosions);
(c) Percentage of subjects with a peak eosinophils/HPF number <1 and <15 at Weeks 12, 26, and 52;
(d) Change from Baseline Global EoE Symptom Score assessed prior to randomisation, which was assessed for the 7-day period prior to the following study visits: Weeks 4, 8, 12, 14, 18, 22, 26, 28, 36, 44, and 52;
(e) Dysphagia: Change in the number of dysphagia episodes at baseline (14-day period prior to randomisation) compared with the 14-day period prior to the time point of interest (Weeks 12, 26, and 52);
(f) Change from Baseline 7-day Eosinophilic Oesophagitis Activity Index (EEsAI) total score assessed prior to randomisation to those assessed at Weeks 12, 26, and 52; (g) Change from Baseline 7-day EEsAI sub-scores to those assessed at Weeks 12, 26, and 52;
(h) Percentage of subjects with mean 7-day EEsAI total score <20 to those assessed at Weeks 12, 26, and 52;
(i) Change from Baseline Patient Global Impression of Severity (PGIS) assessed prior to randomisation at Weeks 4, 8, 12, 14, 18, 22, 26, 28, 36, 44, and 52;
(j) Patient Global Impression of Change (PGIC) at Weeks 4, 8, 12, 14, 18, 22, 26, 28, 36, 44, and 52;
(k) Assessment of treatment failure and relapse, including: Percentage of non-responders by dose at Weeks 12, 26, and 52;
(l) Percentage of subjects requiring emergency endoscopic food dis-impaction by dose before Week 14, between Week 14 and Week 28, and between Week 28 and Week 52; and Percentage of subjects requiring oesophageal dilation by dosing group and part of the study.

Exploratory efficacy endpoints were also analyzed. Exploratory efficacy endpoints include:
(a) Change from Baseline in dysphagia-free days during the 14-day period prior to the following study visits: Weeks 12, 26, and 52;
(b) EoE sustained response (dysphagia): Percentage of all subjects who met the dysphagia secondary endpoint at Week 12 and maintained a dysphagia-related response at Week 26 and Week 52;
(c) Evaluation of PK/PD (cortisol) and exposure-response (efficacy) relationships; Subject's assessment of symptoms compared with the previous visit at Weeks 4, 8, 12, 14, 18, 22, 26, 28, 36, 44, 52, and the Early Termination Visit (if applicable); and
(d) Evaluation of Health-Related Quality of Life (HRQoL) based on the Adult Eosinophilic Esophagitis Quality of Life Questionnaire (EoE-QoL-A) at randomisation, Week 12, Week 26, Week 52 for all subjects by dose and subgroup.

Subjects receiving single-blind (to subject) treatment (3 mg twice daily [BID] in Part 2) will be tabulated separately: Percentage of subjects who were classified as histologic non-responders at Week 12 and have ≤6 peak eosinophils/HPF at all biopsied oesophageal locations at Week 26 and Week 52; Change from Baseline dysphagia episodes during the 14-day period prior to Week 26 and Week 52 for subjects who were classified as non-responders at Week 12; Percentage of subjects who were classified as histologic non-responders at Week 12 and meet the primary endpoint at Week 26 and Week 52.

A scoring structure, and various endpoints was derived from the PROSE; Psychometric measurement properties of the PROSE were evaluated; Anchor and distribution analyses to evaluate meaningful changes on the PROSE.

Safety endpoints were measured such as Frequency of treatment-emergent adverse event (TEAEs); TEAEs leading to discontinuation; Treatment-emergent serious adverse events (SAEs); Percentage of subjects with serum cortisol level ≤5 μg/dL (≤138 nmol/L) or abnormal adrenocorticotropic hormone (ACTH) stimulation test (serum cortisol <16 μg/dL [≤440 nmol/L] at 60 minutes); The number of subjects discontinuing for HPA axis suppression will be recorded; and frequency of oral and esophageal candidiasis.

Part 1 of study SP-1011-002 was completed, and efficacy and safety after 12 weeks of treatment with FP (1.5 mg HS, 1.5 mg BID, 3 mg HS, or 3 mg BID) or placebo are summarized below.

### Results

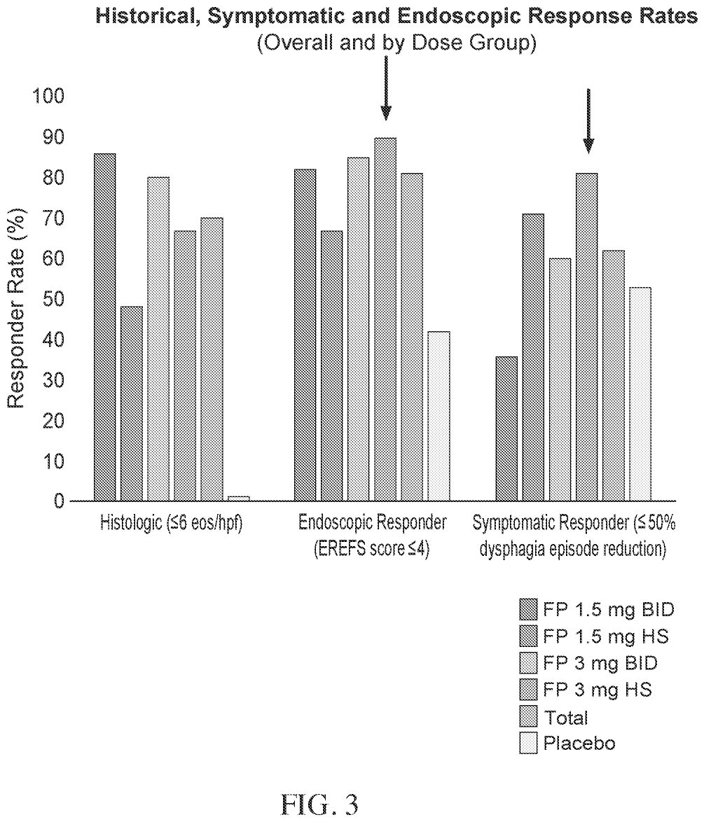

As shown in FIG. 3 and Table C, resolution of EoE features in patients receiving FP had higher histologic (79% vs. 0%; P<. 001) and endoscopic (88% vs. 38%; P<. 001) response rates versus placebo. Surprisingly, a post hoc analysis conducted for the subgroup of patients with strictures and/or grade 2 rings from all dosing groups (i.e., 1.5 mg or 3 mg of FP at bedtime and 1.5 mg or 3 mg of fluticasone twice daily) reported reduced stricture symptoms following the FP treatment as compared to the placebo. Patients receiving FP had lower rates of persistent strictures and/or rings at Week 12 versus placebo: stricture (21% vs. 46%; P=. 075), rings (7% vs. 46%; P <. 001), either stricture or rings (28% vs. 77%; P=. 002), and both stricture and rings (0% vs. 15%). A summary of the response rates at Week 12 is provided in Table D. A summary of the treatment-emergent adverse event (TEAE) occurred during the study is provided in Table E.

TABLE C

| | Dosing Group | | | | | | |
|---|---|---|---|---|---|---|---|
| Subject with Strictures | FP 3 | FP 3 | FP 1.5 | FP 1.5 | | | |
| and/or Grade 2 Rings | mg BID | mg HS | mg BID | mg HS | TOTAL | Placebo | P- |
| at Baseline | (n = 7) | (n = 11) | (n = 12) | (n = 13) | (n = 43) | (n = 13) | value¹ |
| Histology Responder | 86 | 82 | 100 | 54 | 79 | 0 | <0.001 |
| Rate | | | | | | | |
| (≥6 eos/hpf), % | | | | | | | |
| Endoscopy Responder | 86 | 100 | 92 | 77 | 88 | 38 | <0.001 |
| Rate (overall EFEFS | | | | | | | |
| score ≤4), % | | | | | | | |
| Symptoms Responder | 57 | 73 | 42 | 77 | 63 | 69 | 0.674 |
| Rate (≤50% reduction | | | | | | | |
| dysphagia episodes), % | | | | | | | |
| Stricture at Week 12, % | 29 | 9 | 42 | 8 | 21 | 46 | 0.075 |
| Stricture and Grade 2 | 0 | 0 | 0 | 0 | 0 | 15 | 0.009 |
| Rings at Week 12, % | | | | | | | |
| Stricture or Grade 2 | 29 | 18 | 50 | 15 | 28 | 77 | 0.002 |
| Rings at Week 12, % | | | | | | | |
| Subjects with new | 0 | 0 | 0 | 0 | 0 | 8 | 0.355 |
| strictures and/or rings | | | | | | | |
| at Week 12, % | | | | | | | |

TABLE D
Response Rates and Outcomes in Subjects with Stricture/Rings

| | FP | |
|---|---|---|
| | Treatment | Placebo |
| Stricture at Week 12, % | 21 | 46 |
| Grade 2 Rings at Week 12, % | 7 | 46 |
| Stricture or Grade 2 Rings at | 28 | 77 |
| Week 12, % | | |
| Stricture and Grade 2 Rings | 0 | 15 |
| at Week 12, % | | |

TABLE E
Treatment-emergent adverse event (TEAE) occurred during the study

| | Randomized Dosing Group | | | | | | | | | | |
|---|---|---|---|---|---|---|---|---|---|---|---|
| | FP 3 | | FP 3 | | FP 1.5 | | FP 1.5 | | | | |
| Treatment-Emergent | mg BID | | mg HS | | mg BID | | mg HS | | Placebo | | Total FP |
| Adverse Event (TEAE) | (n = 20), | | (n = 21), | | (n = 23), | | (n = 21), | | (n = 20), | | (n = 85), |
| TEAE Category | n (%) | | n (%) | | n (%) | | n (%) | | n (%) | | n (%) |
| At least 1 TEAE, n (%) | 17 | (85) | 16 | (76) | 17 | (74) | 13 | (62) | 13 | (65) | 63 (74) |
| Maximum severity of TEAE | | | | | | | | | | | |
| Mild, n (%) | 12 | (60) | 16 | (29) | 14 | (61) | 11 | (52) | 10 | (50) | 43 (51) |
| Moderate, n (%) | 5 | (25) | 9 | (43) | 3 | (13) | 1 | (5) | 3 | (15) | 18 (21) |
| Severe, n (%) | 0 | | 1 | (5) | 0 | | 1 | (5) | 0 | | 2 (2) |
| TEAE related to study | 10 | (50) | 4 | (29) | 5 | (22) | 2 | (10) | 4 | (20) | 21 (25) |
| drug, n (%) | | | | | | | | | | | |
| TEAE leading to study | 1 | (5) | 0 | | 2 | (9) | 0 | | 2 | (10) | 3 (4) |
| discontinuation, n (%) | | | | | | | | | | | |
| Serious TEAE, n (%) | 0 | | 1 | (5) | 0 | | 0 | | 0 | | 1 (1) |
| TEAE resulting in death, | 0 | | 0 | | 0 | | 0 | | 0 | | 0 |
| n (%) | | | | | | | | | | | |
| TEAE of special interest, | 7 | (35) | 1 | (5) | 3 | (13) | 0 | | 0 | | 11 (13) |
| n (%) | | | | | | | | | | | |

Abbreviations: BID = twice daily; HS-hora somni (at bedtime); N = number.
Note:
TEAE = any adverse event that started or worsened in severity after the first dose of study drug.
Note:
For maximum severity rows, if a subject had more than 1 TEAE, they were counted only once based on the maximum severity.

### CONCLUSIONS

The data suggest that an ODT comprising FP at the 3-mg HS (bedtime) dose provides the best balance of safety and efficacy for inducing histologic remission, symptomatic improvement over 52 weeks, with lower rates of candidiasis. Further, once daily dosing at bedtime has the potential to encourage better treatment compliance. In a post-hoc analysis of patients with strictures and/or grade 2 esophageal rings at baseline, FP demonstrated improvement or resolution of these features in most patients, particularly for the 3-mg HS dosing group. This is an important outcome as fibrostenosis has been linked to lower treatment responses. The results indicate that FP will be effective in treating strictures, including severe strictures, and rings in a variety of conditions.

### Example 2. Diagnosis of Esophageal Stricture

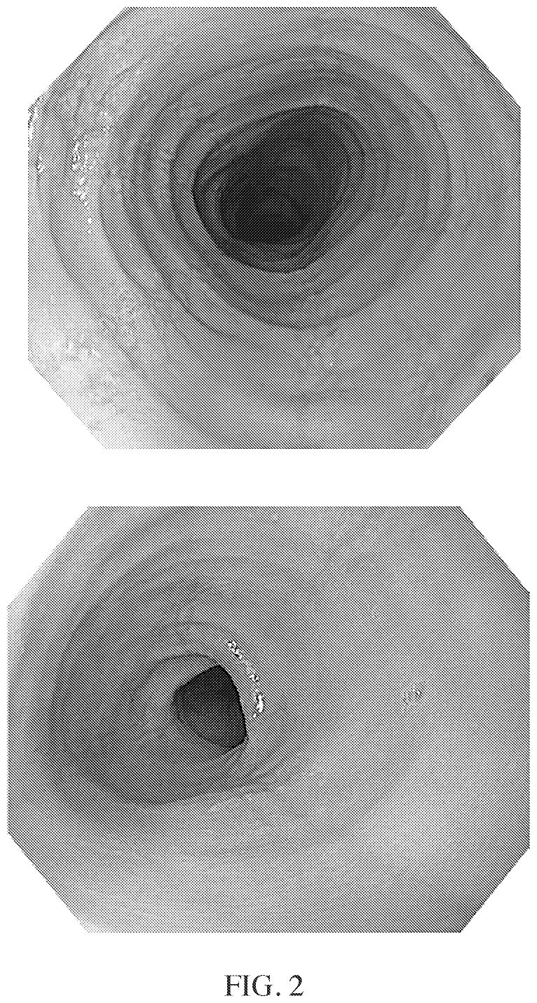

Esophageal stricture is detected in a patient by endoscopy (EGD) and/or performing Modified Barium Swallow Study (MBSS). The EGD is performed with a long, flexible tube with about 9 mm in diameter that is attached to a video camera at the end of the scope. Prior to insertion of the tube, the patient may choose to undergo minimal sedation, moderate sedation, deep sedation or general anesthesia. During the EGD, the tube is introduced into the mouth of the patient and maneuvered into the esophagus, stomach and intestine. Sample collection for biopsy may be performed when stricture, rings, or other abnormal esophageal symptoms are detected. The esophageal stricture or ring is detected visually using a video display on the endoscope, as shown in FIG. 2. If the endoscope cannot pass through the patient's esophagus, the patient is considered to have severe esophageal stricture.

For the Modified Barium Swallow Study (MBSS), the patient sits in a chair and swallows food or liquid that includes barium and is monitored by a physician or medical professionals. The swallowed barium material is visualized on X-rays, and the movement of the swallowed food or liquid is recorded and analyzed.

### Example 3. Diagnosing and Treating a Patient Suffering from Esophageal Stricture

A method of topically treating esophageal stricture is described. Firstly, a patient will be diagnosed by a physician observing the presence or symptoms of esophageal stricture after inserting an endoscope into the patient's esophagus. The endoscope will be attached to a camera or video to instantly visualize the oesophagus lumen.

Esophageal stricture is detected when the endoscope does not readily pass through the esophagus. When the endoscope fails to pass through the proximal to the distal portion of the esophagus, the patient is diagnosed with a severe esophageal stricture. Following the diagnosis of the esophageal stricture, the patient is orally administered 1.5 mg or 3 mg of fluticasone propionate in an orally disintegrating tablet prior to bedtime.

1. A method of topically treating an esophageal stricture in a patient in need thereof with a corticosteroid, comprising:
(a) detecting the esophageal stricture; and then
(b) orally administering the corticosteroid,
wherein a therapeutically effective amount of the oral corticosteroid contacts the esophageal stricture, thereby reducing the severity of the esophageal stricture.

2. The method of claim 1, wherein the detecting in step (a) comprises inserting an endoscope into the patient's esophagus.

3. The method of claim 2, wherein the endoscope has a diameter of about 8-10 mm, or about 9 mm.

4. The method of claim 2 or 3, wherein the esophageal stricture is detected visually using a video display on the endoscope.

5. The method of claim 2 or 3, wherein the esophageal stricture is detected because the endoscope cannot pass through the patient's esophagus.

6. The method of any one of the preceding claims, wherein the patient also has a ring.

7. The method of claim 6, wherein the ring is a grade 2 or grade 3 ring.

8. The method of claim 7, wherein administrating the corticosteroid reduces the grade of the ring by at least 1 grade.

9. The method of claim any one of preceding claims, wherein the corticosteroid is administered while the patient is lying down or immediately prior to the patient lying down.

10. The method of claim 9, wherein the lying down is in a supine position.

11. The method of claim 9, wherein the oral corticosteroid is administered within about 5, 4, 3, 2, or 1 minutes prior to the patient lying down.

12. The method of claim 1, wherein the oral corticosteroid is administered about 30 minutes or less before target sleep time.

13. The method of any one of the preceding claims, wherein the corticosteroid is administered once daily.

14. The method of any one of the preceding claims, wherein corticosteroid has a systemic bioavailability of less than or equal to about 20% of its dose.

15. The method of any one of the preceding claims, wherein the corticosteroid is budesonide, fluticasone, flunisolide, ciclesonide, mometasone or beclomethasone, or a pharmaceutically acceptable salt, solvent, ester, polymorph or prodrug thereof.

16. The method of any one of the preceding claims, wherein the corticosteroid is budesonide, fluticasone, or a pharmaceutically acceptable ester thereof.

17. The method of any one of the preceding claims, wherein the corticosteroid is fluticasone propionate.

18. The method of any one of the preceding claims, wherein the corticosteroid is formulated as a solid composition.

19. The method of claim 18, wherein the solid composition is in the form of a gel, lozenge, lollipop, effervescent tablet, powder, granules or an orally disintegrating composition.

20. The method of claim 19, wherein the orally disintegrating composition is a tablet, wafer, film, or lyophilized matrix.

21. The method of claim 20, wherein the orally disintegrating composition is a tablet.

22. The method of claim 21, wherein the tablet comprises:
a. the corticosteroid in an amount of from about 1.0 mg to about 7.5 mg;
b. a pharmaceutically acceptable carrier combined with the corticosteroid; and
c. rapidly dispersing microgranules,
wherein the orally disintegrating tablet disintegrates within 60 seconds when tested using the USP <701> method for disintegration time.

23. The method of any one of the preceding claims, wherein the corticosteroid provides an average maximum blood plasma concentration (Cmax) of less than or equal to about 500 pg/mL after oral administration of about 0.5 mg to about 20 mg of the oral corticosteroid.

24. The method of any one of the preceding claims, wherein the corticosteroid provides an average AUC₀₋₂₄ of less than or equal to about 3,000 pg*h/mL after oral administration of about 0.5 mg to about 20 mg of the oral corticosteroid.

25. The method of any one of the preceding claims, wherein the patient has a Cmax of the corticosteroid of less than or equal to about 200 pg/mL following oral administration of about 1 mg to about 7.5 mg of the corticosteroid.

26. The method of claim 1, wherein the patient has eosinophilic esophagitis (EoE), gastroesophageal reflux disease (GERD), non-erosive reflux disease (NERD), erosive esophagitis, Barrett's esophagus, eosinophilic gastroenteritis, hypereosinophilic syndrome, corrosive chemical esophagitis, radiation-induced esophagitis, chemotherapy-induced esophagitis, transient drug-induced esophagitis, persistent drug-induced esophagitis, Crohn's disease of the esophagus, post-surgical resection of the esophagus; or pseudomembranous esophagitis.

27. The method of any one of the preceding claims, wherein the corticosteroid is administered for at least 1, 2, 3, 4, 5, 6, 7, 8, 9, 10, 11, or 12 weeks, or 1, 2, 3, 4, 5, 6, 7, 8, 9, 10, 11, or 12 months.

28. The method of any one of the preceding claims, wherein after administration of the corticosteroid, the patient exhibits an improvement in symptoms, as measured a reduction in eosinophil count, an increase in dysphagia-free days, a reduction in episodes of dysphagia, improvement in EREFS score, EndoFLIP documentation of improved esophageal compliance, evaluation of biomarkers, a decrease in episodes of food impaction, an improvement in EEsAI scores (patient, physician, endoscopy, pathology scores), EoE-QOL-A, Visual Dysphagia Questionnaire (VDQ), Avoidance Modification and Slow Eating (AMS) scores, or histology, at least one symptom score measured using a patient reported outcome symptom evaluation (PROSE) instrument after an episode of dysphagia, Global EoE score, patient global impression of severity (PGIS), or patient global impression of change (PGIC).

29. The method of any one of the preceding claims, wherein the patient has EoE.

30. The method of any one of the preceding claims, wherein the patient's risk of candidiasis is less than about 10%.

31. The method of claim 30, wherein the patient's risk of candidiasis about 5% or less.

32. The method of claim 30 or 31, wherein the candidiasis is oral candidiasis or esophageal candidiasis.

33. The method of claim 32, wherein the candidiasis is oral candidiasis.

34. The method of claim 33, wherein the patient's risk of oral candidiasis is less than about 4%, less than about 3%, less than about 2%, or less than about 1%.

35. The method of any one of claims 30-32, wherein the candidiasis is esophageal candidiasis.

36. The method of claim 35, wherein the patient's risk of esophageal candidiasis is about 4.8%.

37. The method of any one of preceding claims, wherein eosinophil count in the patient's esophagus is reduced compared to the patient's baseline eosinophil levels.

38. The method of claim 37, wherein the eosinophil count is reduced to no more than 6 eosinophils per high power field (hpf).

39. The method of claim 38, wherein the eosinophil count is measured in the distal portion of the esophagus, the proximal portion of the esophagus, or both.

40. The method of claim 39, wherein eosinophil count in the distal portion of the esophagus is no more than 6 eosinophils per hpf.

41. The method of claim 39, wherein eosinophil count in the proximal portion of the esophagus is no more than 6 eosinophils per hpf.

42. The method of any of the preceding claims, wherein the EREFS score is improved by about 0.3 to 1.5 points.

43. The method of any one of the preceding claims, wherein the EREFS score is less than or equal to 4 points.

44. The method of any one of the preceding claims, wherein the number of dysphagia episodes is reduced compared to a baseline period prior to treatment.

45. The method of any one of the preceding claims, wherein the number of dysphagia episodes is reduced by at least 50% compared to a baseline period prior to treatment.

46. The method of any one of the preceding claims, wherein the corticosteroid is fluticasone propionate, and it is administered at a dose ranging from about 3 mg to about 6 mg.

47. The method of any one of the preceding claims, wherein corticosteroid is administered at an equipotent dose of about 3-6 mg fluticasone propionate.

48. The method of any one of the preceding claims, wherein the corticosteroid is fluticasone propionate, and it is administered at a dose ranging from about 1.5 mg to about 3 mg.

49. The method of claim 48, wherein the dose of fluticasone propionate is about 1.5 mg to about 3 mg.

50. The method of any one of the preceding claims, wherein the patient developed no new strictures while being treated with the corticosteroid.

51. The method of any one of the preceding claims, wherein the patient developed no new rings while being treated with the corticosteroid.

52. The method of claim 46-51, wherein the fluticasone propionate is administered once daily.

53. The method of claim 52, wherein the fluticasone propionate is administered at bedtime.

54. The method of claim 52, wherein the corticosteroid is administered for at least 12 weeks, and after 12 weeks, the endoscope passes through the patient's esophagus.
